(19) United States
(12) Patent Application Publication
(10) Pub. No.: US 2023/0159914 A1
(43) Pub. Date: May 25, 2023

(54) METHODS FOR RECONSTRUCTING SINGLE CELL GENOME

(71) Applicant: Digenomix Corporation, Foster City, CA (US)

(72) Inventors: Lei Xi, Foster City, CA (US); Patrick Leong, Palo Alto, CA (US); Aleksandar Mihajlovic, New York, NY (US)

(73) Assignee: Digenomix Corporation, Foster City, CA (US)

(21) Appl. No.: 17/848,944

(22) Filed: Jun. 24, 2022

Related U.S. Application Data
(60) Provisional application No. 63/214,723, filed on Jun. 24, 2021.

(51) Int. Cl. C12N 15/10; C12Q 1/6806

(52) U.S. Cl. CPC C12N 15/1065; C12N 15/1082; C12Q 1/6806

(57) ABSTRACT

Single-cell sequencing provides a new level of granularity in studying the heterogeneous nature of cancer cells. For some cancers, this heterogeneity is the result of copy number changes of genes within the cellular genomes. The ability to accurately determine such copy number changes is critical in tracing and understanding tumorigenesis. Current single-cell genome sequencing methodologies infer copy numbers based on statistical approaches followed by rounding decimal numbers to integer values. Such methodologies are sample dependent, have varying calling sensitivities which heavily depend on the sample's ploidy and are sensitive to noise in sequencing data. Described herein are novel methods for reconstructing the genome of a single cell. The methods comprise fragmenting the genome using a loaded transposase, linking together fragments based on the overlapping 8-10 nucleotide genomic sequence immediately next to the transposon end to restore the order of the fragments as originally present in the genome, and reconstructing the genome by disregarding fragments that result from a defective transposase reaction and therefore cannot be linked with a neighboring fragment.

### CROSS-REFERENCES TO RELATED APPLICATIONS

This application is a Non-Provisional application which claims the benefit of priority to U.S. Provisional Application No. 63/214,723 filed Jun. 24, 2021, the entire contents of which are hereby incorporated in its entirety for all purposes.

### REFERENCE TO SUBMISSION OF A SEQUENCE LISTING AS A TEXT FILE

The Sequence Listing written in file 102488-1335598-000210US_ST25.txt created on Sep. 30, 2022, 12,946 bytes, machine format IBM-PC, MS-Windows operating system, is hereby incorporated by reference in its entirety for all purposes.

### BACKGROUND OF THE INVENTION

In recent years, attention has been extended from exonic single nucleotide variations to underlying copy number variations of genes (ref. 1). It has been postulated that gene copy number variation is a result of a cellular response intended to increase survivability under stress inducing conditions. Such copy number changes can either promote survival of the cell under adverse conditions, or can induce abnormal behavior resulting in carcinogenesis, metastasis, or drug resistance (refs. 2-6). The dynamics and heterogeneity of genomic copy number changes in response to adverse stresses has been the focus of many investigations to elucidate the forces that shape genomes in eukaryotic cells and likely influence karyotypic evolution in cancer cells (refs. 4-7).

Traditionally, molecular cytogenetic techniques such as fluorescence in situ hybridization (FISH) and spectral karyotyping (SKY) are the chosen methods to determine discrete gene copy numbers (refs. 8, 9). However, these techniques suffer from low throughput, low resolution, high labor cost, and often higher error rates (ref. 10). More recently, several NGS and NGS-based single-cell DNA sequencing technologies with improved throughput have been reported (ref 11). The copy number of the genes are calculated in secondary analyses by binning the number of mapped reads across the genome, grouping the bins into segments of similar quantities, then using these non-integer values and statistical methods to infer a discrete copy number for each segment. The performance of these algorithms is determined by their tuning parameters, sequencing noise, and the ploidy of genomes. As a result, the precision can range from 0% to 90% in the worst cases using simulated data (ref 12). Previously, a novel single-cell DNA sequencing scheme was described (ref 13,14; U.S. Pat. No. 10,526,601), termed Barcodes-In-Genome sequencing (BIGseq).

Single-cell DNA sequencing methods are often treated as bulk sequencing methods that are merely applied to single cell samples. The apparent similarity of the sample preparation and processing procedures shared by single cell and bulk sequencing protocols may reinforce this notion. However, due to the heterogeneity of gene copy numbers in cancer cells and the undefined number of genomes in the bulk sample, the copy number per genome determined by current bulk sequencing protocols may be calculated to be an approximation, i.e. a decimal value that is not necessarily the true value and creates a conundrum to make sense at the biological level. However, copy numbers can be determined discretely when each of the genomes is interrogated individually. Unfortunately, current single cell DNA sequencing technologies fail to capture the discreteness, as they adopt secondary analysis methodology developed for bulk sequencing (12). These statistics-heavy methodologies are not robust; the inferred copy number varies when a different bin size is chosen, or a different segmentation option is selected, or ploidy of the genome deviates from diploid (12).

Described herein are methods and compositions for reconstructing a single cell genome, and for counting discrete gene copy numbers within single cells.

### BRIEF SUMMARY OF THE INVENTION

Described herein are methods and compositions for improving the reconstruction of single cell genomes. In one aspect, the disclosure provides a method for improving the reconstruction of a single cell genome, the method comprising:
(i) obtaining genomic DNA derived from a single fully disrupted cell;
(ii) contacting and fragmenting the genomic DNA using a transposase loaded with two identical transposon ends to form a plurality of genomic DNA fragments each labeled with an identical transposon end at its 5′ and 3′ ends;
(iii) extending a complementary strand of each fragment using a universal primer comprising a sequence complementary to the identical transposon end to generate one or more extension products;
(iv) determining the nucleotide sequence between transposon ends of the extension products;
(v) detecting one or more shorter extension products and one longer extension product comprising one identical segment of genomic sequence;
(vi) disregarding the one or more shorter extension products; and
(vii) identifying appropriate connections that facilitate sequence chaining among the remaining extension products based on overlapping unique 8-10 nucleotide sequences immediately next to the transposon end to determine the phase of each sequence, thereby reconstructing the genome.

In some embodiments, step (vii) comprises determining the phase of the nucleotide sequence between transposon ends of the extension products from step (iv). In some embodiments, step (vii) is performed using a bioinformatics process. In some embodiments, step (vii) determines the ploidy of a genomic region in the genome. The ploidy of a genomic region can be determined by counting the number of phases determined in step (vii).

In another aspect, the disclosure provides a method for linking together two or more DNA fragments that originated from the same DNA molecule, the method comprising:
(i) obtaining genomic DNA derived from a single fully disrupted cell;
(ii) contacting and fragmenting the genomic DNA using a transposase loaded with two identical transposon ends to form a plurality of genomic DNA fragments each labeled with an identical transposon end at its 5′ and 3′ ends;
(iii) extending a complementary strand of each fragment using a universal primer comprising a sequence complementary to the identical transposon end to generate one or more extension products;
(iv) determining the nucleotide sequence between transposon ends of the extension products;
(v) detecting one or more shorter extension products and one longer extension product comprising one identical segment of genomic sequence;
(vi) disregarding the one or more shorter extension products; and
(vii) identifying appropriate connections that facilitate sequence chaining among the remaining extension products based on overlapping unique 8-10 nucleotide sequences immediately next to the transposon end to determine the phase of each sequence, thereby linking together the two or more DNA fragments.

In some embodiments, step (vii) comprises determining the phase of the nucleotide sequence between transposon ends of the extension products from step (iv). In some embodiments, step (vii) is performed using a bioinformatics process. In some embodiments, step (vii) determines the ploidy of a genomic region in the genome. The ploidy of a genomic region can be determined by counting the number of phases determined in step (vii).

In some embodiments, the extension products are linked together downstream (in the 5′ to 3′ direction) by concatenating contiguous fragments at transposon junctions. In some embodiments, the sequenced extension products are linked according to their unique fragment identifiers (UFIs), which comprise sequences that form the transposon junctions on each end of a fragment. In some embodiments, the extension products are linked using bioinformatic analysis.

In some embodiments, the disregarded extension products from step (vi) comprise a sequence complementary to the 5′ end of one or more retained extension product(s). In some embodiments, the disregarded extension products from step (vi) comprise a sequence complementary to the 3′ end of one or more retained extension product(s). In some embodiments, the disregarded extension products from step (vi) comprise sequences complementary to both the 5′ and 3′ ends of one or more retained extension products. In some embodiments, the one or more retained extension products comprise a neighboring or adjacent in-phase extension product. In some embodiments, the one or more retained extension products comprise a nucleic acid sequence that is distinct or different from the nucleic acid sequence of the disregarded extension products from step (vi).

In some embodiments, the method further comprises amplifying the extension products. In some embodiments, the method further comprises adding a barcode sequence to the amplified extension products.

In some embodiments, during bioinformatic analysis the in-silico extension products are linked, concatenating the fragments at transposon junctions based on matching 5′ and 3′ sequences of neighboring contiguous fragments.

In some embodiments, the transposon end comprises a universal primer.

In some embodiments, the single cell genome comprises one or more alleles of at least one genetic locus. For example, the single cell genome can comprise one or more alleles (i.e., 1, 2, 3, 4 or more alleles) of one or more genetic loci, such as one, two, three, four, five, six, seven, eight, nine, ten, 20, 30, 40, 50, 100 or more genetic loci. In some embodiments, the single cell genome comprises two or more chromosomes.

In some embodiments, the single fully disrupted cell is a monoploid cell, diploid cell, a tetraploid cell, a polyploid cell, or a cancer cell.

In another aspect, the disclosure provides a method for counting two or more DNA molecules. In some embodiments, the method comprises:
(i) obtaining genomic DNA derived from a single fully disrupted cell;
(ii) contacting and fragmenting the genomic DNA using a transposase loaded with two identical transposon ends to form a plurality of genomic DNA fragments each labeled with an identical transposon end at its 5′ and 3′ ends;
(iii) extending a complementary strand of each fragment using a universal primer comprising a sequence complementary to the identical transposon end to generate one or more extension products;
(iv) determining the nucleotide sequence of the extension products;
(v) detecting one or more shorter extension products and one longer extension product comprising one identical segment of genomic sequence;
(vi) disregarding all shorter extension products; and
(vii) identifying appropriate connections that facilitate sequence chaining among the remaining extension products based on overlapping unique 8-10 nucleotide sequences immediately next to the transposon end to determine the phase of each sequence to create a contiguous sequence;
(viii) assigning the contiguous sequence to a first or second DNA molecule, thereby counting the DNA molecules.

In some embodiments, step (vii) comprises determining the phase of the nucleotide sequence of the extension products from step (iv). In some embodiments, the two or more DNA molecules comprise the same or identical nucleic acid sequences. In some embodiments, the assigning step comprises using the rules of exclusivity and greediness. In some embodiments, counting the DNA molecules comprises counting digital DNA molecules.

In another aspect, a system or device for performing one or more steps of the methods described herein is provided. In some embodiments, the system or device is a computer system or computerized device for performing one or more steps of the methods described herein. In some embodiments, the computer system and/or computerized device is configured to implement one or more embodiments of a method described herein.

In another aspect, provided are one or more computer-readable media collectively having stored thereon computer-executable instructions for performing one or more embodiments of the methods described herein. In some embodiments, the computer-executable instructions, when executed with one or more computing systems or computerized devices, can be used to perform one or more of the following steps: determine the nucleotide sequence between transposon ends of an extension product; detect one or more shorter extension products and one longer extension product comprising one identical segment of genomic sequence; disregard the one or more shorter extension products; identify appropriate connections among the remaining extension products based on overlapping unique 8-10 nucleotide sequence immediately next to the transposon end; and/or reconstruct the genome.

In some embodiments, the computer-executable instructions, when executed with one or more computing systems or computerized devices, can be used to perform one or more of the following steps: determine the nucleotide sequence between transposon ends of an extension product; detect one or more shorter extension products and one longer extension product comprising one identical segment of genomic sequence; disregard the one or more shorter extension products; identify appropriate connections among the remaining extension products based on overlapping unique 8-10 nucleotide sequence immediately next to the transposon end to create a contiguous sequence; assign the contiguous sequence to a first or second DNA molecule, and/or count the DNA molecules.

In some embodiments, the computer system and/or computerized device is configured to link together the extension products in 5′ to 3′ order by concatenating neighboring fragments at transposon junctions. In some embodiments, the computer system and/or computerized device is configured to link together the sequenced extension products using their unique fragment identifiers (UFI), which comprise the start and end nucleotide positions of the fragments. In some embodiments, the computer system and/or computerized device is configured to bioinformatically link the extension products by concatenating the fragments at transposon junctions. In some embodiments, the computer system and/or computerized device is configured to

In some embodiments, the computer system and/or computerized device is configured to implement an algorithm described herein. In some embodiments, one or more of steps (iv) (viii) in the embodiments above is performed using a computer algorithm or bioinformatic algorithm. In some embodiments, the algorithm is a bioinformatics algorithm, such as the BIGseq algorithm.

In some embodiments, one or more steps of the methods described herein can be performed by a third party.

### BRIEF DESCRIPTION OF THE DRAWINGS

FIGS. 1A-1E. Archipelagos identified in sample 6Y5j. FIG. 1A. Distribution of identified archipelagos (dark blue bar) is displayed for 16 chromosomes of 6Y5j. The size of each chromosome is labelled with a light blue line above the coordinate. FIG. 1B. Archipelagos of Chr XII/NC_001144.5 are presented. FIG. 1C. An archipelago in the region of Chr XII/NC_001144.5 755,960-775,121 is zoomed in to show fragments after deduplication (SEQ ID NO: 1). FIG. 1D. The 9-base junction and its surrounding regions between Fragments 2 and 3 in FIG. 1C are enlarged. FIG. 1E. Simulation of tagmentation of the same genome region as in FIG. 1C are presented to show the perfect tiling.

FIGS. 2A-2B. GC % of Archipelagos and the junction regions and base preference in the region at the transpososome cutting site. FIG. 2A. GC distribution of Archipelago (blue) and GC distribution of the 9-base junction, at various range of GC %. FIG. 2B. Base preference at 6 positions of top strand surrounding the cutting site for 9-base canonical and 8- and 10-base uncanonical cuts presented by sequence logo. The symmetry of the cutting site is marked by the two boxes. The six bases on the top strand flanking the cutting site in the direction from outside to the inside of the left monomer of the transposase are numbered as: −2, −1, Dup1, Dup2, Dup3 and Dup4, where the cut was made between −1 and Dup1 (SEQ ID NOs: 2-13 respectively, top to bottom).

FIGS. 3A-3G. Defective transpososomes leads to artifacts. FIG. 3A. Nineteen fragments in the archipelago of Chr V/NC_001137.3 22,504 to 27,231 are reconstructed into one molecule. The seemingly lone Fragment 4 led to the discovery of defective transpososomes. FIG. 3B-G show Defective Scenarios 0/1, I/O, and Complex Scenarios 00/11, 11/00, 01/1/0, and 10/01 respectively.

FIGS. 4A-4C. The BIGseq algorithm to identify two molecules. FIG. 4A. Boundary of an archipelago is set. FIG. 4B. Two molecules and redundant fragments are called. FIG. 4C. Final presentation of two molecules.

FIG. 5. Leftover primer sequence. Sequences 1-10 are the reverse reads for Fragment 19 with coordinates 658,201-658,361 of Chr XIV/NC_001146.8, while Sequences 11-18 are the forward reads for Fragment 20 (658,350-658,629). The subsequence CAG from 11 to 18 at the very 5′ end are not present in the reference genome or Fragment 19. They are the last three bases from primers.

FIG. 6. Examples of Complex Scenarios of 00/11 exhibited by Fragments 1-3 (FIG. 6A), 11/00 exhibited by Fragments 1-3 (FIG. 6B), 01/10 exhibited by Fragments 1-3 (FIG. 6C), and 10/01 exhibited by Fragments 1 and 2 (FIG. 6D) respectively.

### DEFINITIONS

Terms used in the claims and specification are defined as set forth below unless otherwise specified.

The term “comprises” is inclusive or open-ended and does not exclude additional, un-recited elements, features or steps.

The term “nucleic acid” refers to a nucleotide polymer. It includes both double- and single-stranded molecules. A double-stranded nucleic acid need not be double-stranded along the entire length of both strands. Some double-stranded nucleic acid are joined with single stranded nucleic acid.

The term “transposon” refers to a nucleic acid molecule that is capable of being incorporated into a nucleic acid by a transposase enzyme. A transposon includes two transposon ends (also termed “arms”) linked by a sequence that is sufficiently long to form a loop in the presence of a transposase. Transposons can be double-, single-stranded, or mixed, containing single- and double-stranded region(s), depending on the transposase used to insert the transposon. For Mu, Tn3, Tn5, Tn7 or Tn10 transposases, the transposon ends are double-stranded, but the linking sequence need not be double-stranded. In a transposition event, these transposons are inserted into double-stranded DNA.

The term “transposon end” refers to the sequence region that interacts with a transposase. The transposon ends are double-stranded for transposases Mu, Tn3, Tn5, Tn7, Tn10, etc. Transposon ends are two short nucleotide sequences that are not connected to each other, in contrast to a complete transposon, where the ends are linked by a sequence (see above). A double-stranded transposon end exhibits two complementary sequences consisting of a “transferred transposon end sequence” or “transferred strand” and a “non-transferred transposon end sequence,” or “non-transferred strand.” Different transposases bind to transposon ends that differ in length and sequence. For example, Tn5 transposase binds to a transposon end comprising 17 to 19 nucleotides.

The term “artificial transposon end” refers to a transposon end in which one or more positions in a wild-type transposon end have been substituted with one or more different nucleotides. In other cases, extra nucleic acids are covalently linked to the transposon end. An example is the Nextera artificial transposon end offered by Illumina.

The term “transposase” refers to an enzyme which is protein in nature, that binds to transposon ends and catalyzes their linkage to other double- or single-stranded nucleic acids, such as genomic DNA. Transposases usually comprise an even-number of subunits and bind two transposon ends. The two transposon ends can be of identical sequence or of different sequences.

Tn5 transposase is a bacterial enzyme that integrates a DNA fragment into genomic DNA. Tn5 transposases bind to 19-bp inverted ends (the outside end sequences) of Tn5 transposons. Only the outside end sequences and Tn5 transposases are required for transposition in vitro. See, Li N, et al. “Tn5 Transposase Applied in Genomics Research.” Int J Mol Sci. 2020; 21(21):8329. Published 2020 Nov. 6. doi:10.3390/ijms21218329. The canonical amino acid sequence of Tn5 transposase from E. coli (also referred to as “tnpA” or “TnP”) is shown in the informal sequence listing. See the internet at www.uniprot.org/uniprot/Q46731. Variants of Tn5 have been engineered that have increased activity, including variants comprising E54K, M56A, and/or L372P mutations. See Picelli S. et al., “Tn5 transposase and tagmentation procedures for massively scaled sequencing projects,” Genome Res. 2014 24: 2033-2040.

The term “transposon junction” refers to the shared, i.e. overlapping sequence identified to be shared by two neighboring fragments resulted from transposition. The common sequences are created by the single transposition event by a single transpososome on the target nucleic acid. The transpososome makes staggered cuts on the top and the bottom strands and link each of the two complementary strands covalently respectively to one strand of two transposon ends of the transpososome. After repairing, the single stranded nucleic acid ends are converted to double stranded, and each resultant fragment shares identical sequences, which is called a junction, except that the upstream fragment has the junction at its 3′ end while the downstream fragment has the junction at its 5′ end. Tn5 has canonical 9-base “junction.”

As used herein, the terms “barcode sequence” and “index sequence” are used to refer to nucleotide sequences that encode information. For example, a “transposon barcode sequence” can identify a particular transposon. An “index sequence” can identify, e.g., the source of the sample nucleic acids under analysis, such as nucleic acids from a particular sample or a particular reaction. Barcodes can be used to distinguish different cells, different treatments, different time points, different positions in space, etc.

“UMI” is an acronym for “unique molecular identifier,” also referred to as “molecular index.” UMIs can be added to sequencing libraries before any PCR amplification steps, enabling the accurate bioinformatic identification of PCR duplicates (see the internet at dnatech.genomecenter.ucdavis.edu/faqs/what-are-umis-and-why-are-they-used-in-high-throughput-sequencing/#:˜:text=UMI %20 is %20an %20acronym %20 for,“%20or%20”Random%20Barcodes”). A UMI is one in a group of indexes in which each index (or barcode) has an index sequence that is different from any of the other indexes in the group. One way to achieve this “uniqueness” is to use a string of nucleotides. For example, if the length of this string is 10 bases, there are more than 1 million unique sequences; if it is 20 bases long, there will be 1012 unique sequences. The string of nucleotides can be synthetic, derived from a natural sequence, or a combination of both. Take GAPDH encoding gene from Assembly GRCH38.p13 for example, which spans from genome coordinate 6534517 to 6538371 on Chromosome 12. A fragment generated by transposition which is mapped from 6534517 to 6534616 can be treated as a UMI consisting of the string of 100 bases from 6534517 to 6534616. A second fragment mapped from 6534520 to 6534600 can be treated as a UMI consisting of the string of 81 bases from 6534520 to 6534600. Thus, alleles of identical sequence can be distinguished by different UMIs generated by random transposition.

“Amplification” according to the present disclosure encompasses any means by which at least a part of at least one target nucleic acid is reproduced, typically in a template-dependent manner, including without limitation, a broad range of techniques for amplifying nucleic acid sequences, either linearly or exponentially. In some embodiments, the amplification is uniform and synchronized. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

“Whole genome amplification” (“WGA”) refers to any amplification method that aims to produce an amplification product that is representative of the genome from which it was amplified. Illustrative WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

The term “qPCR” is used herein to refer to quantitative real-time polymerase chain reaction (PCR), which is also known as “real-time PCR” or “kinetic polymerase chain reaction.”

The term “extension product” refers to a double stranded DNA molecule consisting of two complementary strands. The two complementary strands can be produced by extending a nick in each strand in a template-dependent manner to produce or synthesize a complementary strand. The extension product can be amplified using a primer, e.g., a universal primer, that binds to a primer binding site introduced by tagmentation of the double stranded DNA.

The term “unique fragment identifier” (UFI) refers to a string of nucleotide sequence that is used to identify a fragment. The nucleotide sequence can be the sequence of the fragment itself, or one or more exogenous sequences that are attached to the fragment, for example by ligation, or by tagmentation. The UFI can also comprise combined sequences of the fragment itself and exogeneous sequences. In a simple form, a UFI can be assigned to a fragment using the nucleotide sequence of the 5′ and 3′ ends relative to a reference sequence. Although under some circumstances, the UFI may not be unique if the fragment can be perfectly mapped to more than one place in a targeted genome, the simplicity of UFI can be desirable. UFI's can be used to link neighboring fragments that are produced by transposases. The UFI can also be used as a barcode at the individual fragment level.

The term “neighboring fragment” refers to two fragments that abut each other and are derived physically from one strand of double stranded DNA. In post-sequencing analysis, two fragments are considered neighboring fragments if the nucleotide sequence at the 3′ end of a first fragment is mapped immediately upstream and overlaps the 5′ end of the nucleotide sequence at the 5′ end of a second mapped fragment. Neighboring fragments thus share a common junction at one but not both ends.

The term “transpososome” refers to a nucleoprotein complex involving the transposon ends and a transposase that carries out a transposition reaction.

The term “tagmentation” refers to a method for fragmenting DNA and adding a tag or adapter that is useful for downstream analysis. Tagmentation is described, for example, in Molecular Biology, Third Edition, 2019, David P. Clark, Nanette J. Pazdernik, Michelle R. McGehee; Academic Press. Tagmentation is a man-made transposition carried out by man-made transpososome.

The term “identifying appropriate connections” refers to reconstructing a stretch of genomic DNA sequence by mapping and connecting the matching ends of neighboring fragments. The matching ends can be connected based on the junction generated by a transpososome during a tagmentation reaction.

When used with reference to a cell, the term “diploid” refers to having two sets of unpaired chromosomes. When used with reference to a genetic locus or segment, the term “diploid” refers to the presence of that locus or segment in two copies.

When used with reference to a cell, the term “haploid” refers to having a single set of unpaired chromosomes. When used with reference to a genetic locus or segment, the term “haploid” refers to the presence of that locus or segment in one copy only.

An organism or cell may have one or more chromosomes in excess of the haploid number or of an exact multiple of the haploid number characteristic of the species, which is referred to as “hyperploidy.” The result is one or more unbalanced sets of chromosomes, which are referred to as “hyperdiploid,” “hypertriploid,” “hypertetraploid,” and so on, depending on the number of multiples of the haploid number they contain.

An organism or cell may have fewer than the haploid number or than an exact multiple of the haploid number of chromosomes characteristic of the species. These one or more unbalanced sets of chromosomes are referred to as “hypodiploid,” “hypotriploid,” “hypotetraploid,” and so on, depending on the number of multiples of the haploid chromosomes they contain.

Any deviation from an exact multiple of the haploid number of chromosomes, whether fewer or more, is termed “aneuploidy.” Aneuploidy is consistently observed in virtually all cancers. Somatic mosaicism occurs in virtually all cancer cells, including trisomy 12 in chronic lymphocytic leukemia (CLL) and trisomy 8 in acute myeloid leukemia (AML). Aneuploid cancer cells may have hypoploidy for some chromosomes, while hyperploidy for others.

As used herein, the term “haplotype” refers to a combination of loci at adjacent locations on a chromosome that are physically linked together through deoxyribonucleic acid backbone. A translocation or chromothripsis generate new haplotypes that did not exist before the event.

As use herein, the term “variation” is used to refer to any difference. A variation can refer to a difference between individuals or populations. A variation encompasses a difference from a common or normal situation. Thus, a “copy number variation” or “mutation” can refer to a difference from a common or normal copy number or nucleotide sequence. Other types of variation include those arising from changes in chromosome structure, as in the case of translocation or chromothrepsis and the combination of both. An “expression level variation” or “splice variant” can refer to an expression level or RNA or protein that differs from the common or normal expression level or RNA or protein for a particular cell or tissue, developmental stage, condition, etc.

“Chromothripsis” refers to the phenomenon by which up to thousands of clustered chromosomal rearrangements occur in a single event in localized and confined genomic regions in one or a few chromosomes, and which is known to be involved in both cancer and congenital diseases.

The term “phase” or ‘in-phase” when used with reference to a nucleic acid sequence refers to DNA fragments that originated from one or the same molecule, such as the same genomic DNA molecule.

The term “sequence chaining” refers to a bioinformatics process or algorithm in which two or more fragments are linked together based on the transposon junction sequence shared by the neighboring fragments. As understood by a person of ordinary skill in the art, a bioinformatics process refers to applying “informatics” techniques derived from disciplines such as applied mathematics, computer science, and statistics to understand and organize the information associated with biological macromolecules such as nucleic acid sequences (see Luscombe N M, Greenbaum D, Gerstein M. What is bioinformatics? A proposed definition and overview of the field. Methods Inf Med. 2001; 40(4):346-58. PMID: 11552348).

The term “retained” extension product refers to an extension product that is not disregarded during the bioinformatic process of genome reconstruction. Thus, retained extension products may be linked together with other extension products to reconstruct the genome.

The term “remaining extension products” refers to one or more unprocessed extension products that are subsequently processed to identify appropriate connections that facilitate sequence chaining.

### DETAILED DESCRIPTION OF THE INVENTION

The present disclosure provides methods and compositions for reconstructing a single cell genome. The methods use a transposase to insert transposons into genomic DNA before further analysis. The genomic DNA can be derived from a single cell or a defined number of identical cells. The disclosure provides advantages in studying the heterogeneous nature of cancer cells. For some cancers, this heterogeneity is the result of copy number changes of genes within the cellular genomes. The ability to accurately determine such copy number changes is important in tracing and understanding tumorigenesis. The methods and compositions described herein can be used to reliably count the number of DNA molecules in a container or single cell in integers, demonstrating the feasibility to count discrete gene copy numbers within single cells.

In one aspect, the disclosure provides a method for reconstructing a single cell genome. In some embodiments, the methods comprise disrupting a single cell to obtain genomic DNA from the cell, followed by tagmentation of the genomic DNA. The tagmentation step can be performed by contacting genomic DNA with a Tn5 transposase loaded with transposon ends. In some embodiments, the contacting is performed in a reaction mixture under conditions that result in fragmentation of the genomic DNA. Suitable conditions are described in the Examples. In some embodiments, the transposase is loaded with two identical transposon ends. Thus, in some embodiments, tagmentation produces a plurality of genomic fragments where all or substantially all of the fragments are tagged with an identical transposon end at the 5′ and 3′ ends of the fragment.

After the tagmentation step, the genomic DNA fragments can then be amplified for a low number of cycles (e.g., one, two or three cycles). In some embodiments, the genomic DNA fragments are amplified using universal primers that hybridize to primer binding sequences in the transposon ends. In some embodiments, the amplified fragments are further amplified with external sequencing primers and the amplified fragments sequenced as described in the Examples.

In some embodiments, the 5′ and 3′ ends of the genomic fragments are then joined or linked together based on the overlapping 8-10 nucleotide genomic sequence located immediately 3′ or downstream of the transposon end to restore the order of the fragments as originally present in the genome, thereby reconstructing the genome. In some embodiments, the genomic fragments are linked together in the original 5′ to 3′ order on the chromosome by concatenating neighboring fragments at the 8-10 nucleotide overlapping ends (also referred to as transposon junctions).

As noted below, some genomic fragments cannot be matched with a neighboring fragment based on defective cutting of the DNA by one reaction center of the transpososome. However, using the methods described herein, the genome can be accurately reconstructed by disregarding fragments that result from a defective Tn5 reaction.

The methods described herein provide an improvement over existing methods by allowing genomic fragments that are produced by a transposase to be assembled into larger contiguous fragments (referred to as contigs). During a tagmentation reaction, Tn5 transposases typically make staggered cuts on the double stranded substrate DNA and ligate to the transposon ends. However, in some instances, one of the two transpososome reaction centers fails to make a nick in one of the DNA strands to complete tagmentation, such that only one strand is cleaved, whereas the other strand remains intact. Thus, only one strand of DNA is successfully ligated to the transposon end and extended in a template-dependent reaction from the nick. Defective transposition reactions can make one copy of double stranded DNA to produce overlapping fragments that would be confused with the tagmentation result derived from two copies of double stranded DNA, thus making the “exclusive” rule hard to apply in the reconstruction step.

The present disclosure solves this problem by identifying fragments that result from defective tagmentation. In some embodiments, the methods fragment genomic DNA by contacting the genomic DNA with a loaded transposase. In some embodiments, the transposase is loaded with two identical transposon ends, and tagmentation produces a plurality of genomic fragments, where all or substantially all fragments are labeled with an identical transposon end at the 5′ and 3′ ends. In some embodiments, the complementary strands of the fragments are extended to generate extension products where the two complimentary strands are of equal length. In some embodiments, the extension products are amplified, for example, by using a primer that binds or hybridizes to a nucleotide sequence (primer binding site) in the transposon ends. The fragments can then be sequenced to determine the nucleotide sequence between transposon ends of the extension products. If the transposase fails to cleave one strand of the substrate DNA, one extra shorter extension product is produced from the complimentary strand. For example, the failed cleavage site results in one strand (e.g., the “plus” strand) being nicked by the transposase and the other, complementary strand (e.g., the “minus” strand) not being nicked. Template-dependent primer extension of the plus strand from the nick results in a first, shorter fragment extending from the nick at the expected cleavage site, and the generation of a second, longer extension product (longer fragment) that encompasses the expected cleavage site on the complementary (or minus) strand where the expected cleavage by the transposase failed to occur. In some embodiments, the shorter extension product comprises a portion of the nucleotide sequence of the longer extension product. If the shorter extension product and the longer extension product comprise an identical segment of genomic sequence, this indicates that the shorter extension product resulted from extension of the plus strand complementary to the minus strand that was not cleaved by the loaded transposase. The shorter and longer fragments can also share a transposon junction. The genome can be reconstructed by joining the longer extension product with adjacent or neighboring fragments based on the matching sequence ends at the transposon junctions to become incorporated into the synthetic, reconstructed genomic sequence. In contrast, the shorter extension product containing a portion of the sequence of the longer extension product can be discarded and not included in the reconstructed genome. If each of neighboring transposases fails to cleave one strand, either on the same strand or different strand, this will result in more shorter extension products that include a portion of the sequence of the complementary longer extension products. If this occurs, the one or more shorter extension products are disregarded when reconstructing the genomic sequence.

After the one or more shorter extension products are disregarded, the method identifies appropriate connections that facilitate sequence chaining among the remaining extension products based on overlapping unique 8-10 nucleotide sequences immediately next to the transposon end (also referred to as “transposon junctions”). Sequence chaining of the remaining extension products can be used to determine the ploidy of the genomic sample. Sequence chaining of the remaining extension products can also be used to determine the phase of the genomic sequence, i.e., to determine if two or more DNA fragments originated from the same genomic DNA molecule. In some embodiments, the methods comprise mapping and identifying transposon junctions so all the DNA fragments can be assigned to phases to determine if the DNA fragments originated from the same DNA molecule. After the transposon junctions are identified and mapped and the sequences chained together to assign the DNA fragments to phases of the genomic sequence (i.e., originated from the same genomic DNA molecule), the ploidy of the genomic sample or region can be determined by counting the phases of genomic sequence in the sample. In some embodiments, sequence chaining and assignment of the DNA fragments to the correct phase is performed bioinformatically, and the genome can be reconstructed and the ploidy determined as described in the Examples.

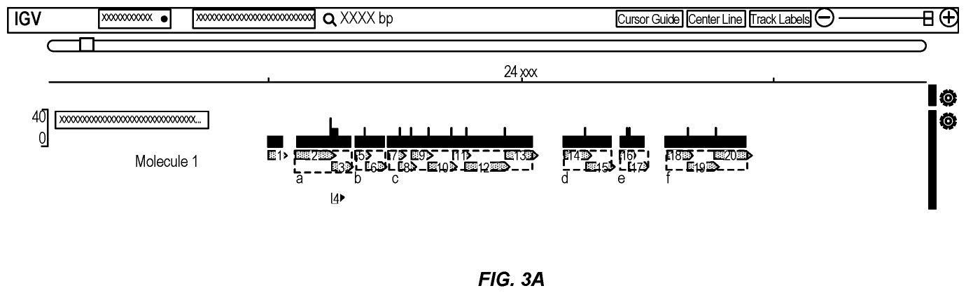

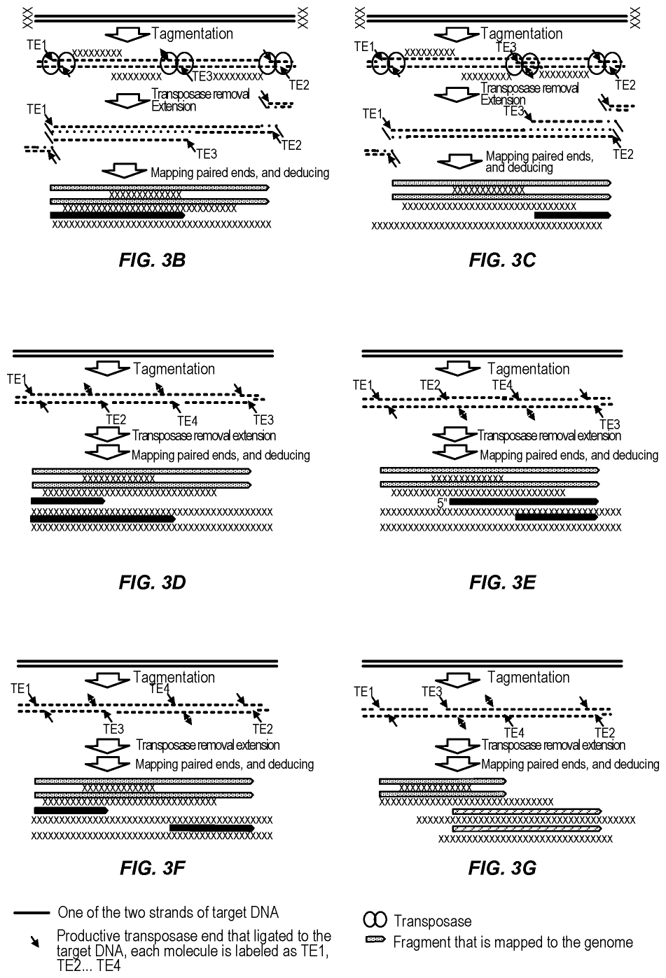

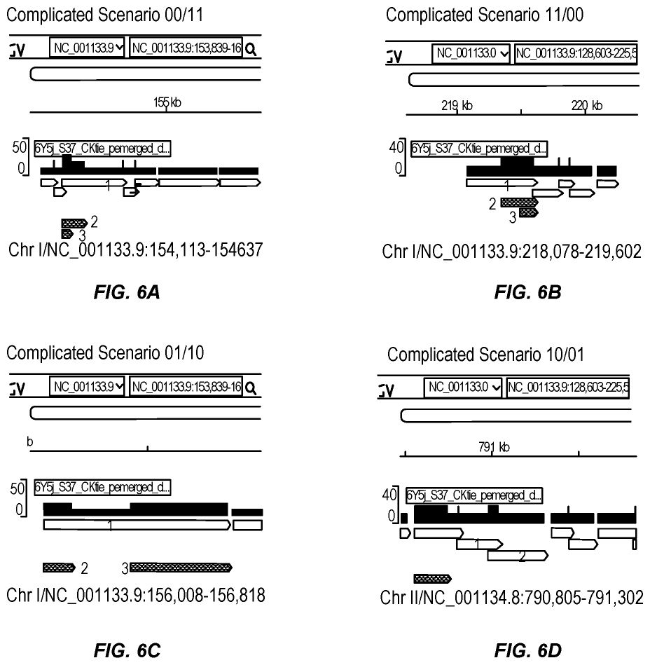

As shown in FIG. 3, the shorter and longer fragments can comprise or share the same 5′ or 3′ ends. In some embodiments, the shorter and longer fragments comprise the same or identical 5′ end (see FIGS. 3B and 3D). In some embodiments, the shorter and longer fragments comprise the same or identical 3′ end (see FIGS. 3C and 3E). In some embodiments, the shorter and longer fragments comprise the same or identical 5′ or 3′ ends (see FIG. 3F). FIG. 3G shows a pattern of fragments generated from two separate defective transpososome reaction centers, where the first defective reaction center is located upstream (5′) on the bottom strand, and the second defective reaction center is located downstream (3′) on the top strand, leading to two overlapping fragments. The scenario in FIG. 3G generates two overlapping fragments, a pattern that is similar to the result if they came from two separate molecules (FIG. 6).

In any of the embodiments described herein, the genomic DNA can be obtained from a single cell or a plurality of substantially identical cells, such as a clonal cell line. In some embodiments, the genomic DNA comprises a monoploid or haploid molecule. In some embodiments, the genomic DNA comprises diploid molecules. In some embodiments, the genomic DNA comprises diploid molecules and one or more extra copies of a genetic locus.

In any of the embodiments described herein, the transposase or transpososome can comprise a wild-type Tn5 transposase, or a mutant, modified or variant Tn5 transposase. In some embodiments, the modified Tn5 transposase has increased activity compared to wild-type Tn5. Non-limiting examples of modified Tn5 transposases include a hyperactive Tn5 transposase from EPICENTRE Biotechnologies, Madison, Wis., USA, and Robust Tn5 Transposase from Creative Biogene Biotechnology, Shirley, N.Y., USA. In some embodiments, the modified or variant Tn5 transposase comprises the E54K, M56A, and/or L372P mutations.

In any of the embodiments described herein, the transposase can loaded with two identical transposon ends. Thus, in some embodiments, the method comprises contacting and fragmenting the genome using a transposase loaded with two identical transposon ends to form a plurality of genomic fragments. In some embodiments, genomic fragments are labeled with an identical transposon end at the 5′ and 3′ ends.

In some embodiments, after the artifactual shorter fragments are discarded, appropriate connections among the remaining extension products are identified based on the overlapping unique 8-10 nucleotide sequence immediately next to the transposon end, and the genome is reconstructed. As noted above, the unique 8-10 nucleotide sequence immediately next to the transposon end is sometimes referred to as a junction.

### Transposases, Transpososomes and Transposition

In one aspect, the methods described herein use a transposase loaded with two identical transposon ends to fragment genomic DNA to generate a plurality of genomic fragments, where all or substantially all fragments are labeled with an identical transposon end at the 5′ and 3′ ends. A “transposition reaction” or “transposition” is a reaction wherein one or more transposon ends are inserted into sample nucleic acids at random sites or almost random sites. Essential components in a transposition reaction are a transposase and DNA oligonucleotides that exhibit the nucleotide sequences of the transposon end, including the transferred transposon end sequence and its complement, the non-transferred transposon end sequence, as well as other components needed to form a functional transposition complex (i.e., a loaded transposase). In some embodiments, the transposase is a loaded Tn5 transposase, or a functional mutant or variant thereof. Suitable transposition complexes for use in the methods described herein include, e.g., a transposition complex formed by a hyperactive Tn5 transposase and a Tn5-type transposon end (Goryshin, I. and Reznikoff, W. S., J. Biol. Chem., 273: 7367, 1998, which is hereby incorporated by reference).

In general, a suitable in vitro transposition system for use in the methods described herein include a transposase enzyme of sufficient purity, sufficient concentration, and sufficient in vitro transposition activity and a transposon end with which the transposase forms a functional complex. Suitable transposase transposon end sequences that can be used in the methods include but are not limited to wild-type or artificial transposon end sequences (see below) that form a complex with a wild-type or mutant transposase. Illustrative transposases include wild-type or mutant forms of Tn5 transposase.

In some embodiments, the transposon end sequences are of the smallest possible size that functions well for the intended purpose, but are large enough that the same sequence is present only rarely or preferably, is not present at all, in the sample nucleic acids. Suitable in vitro transposition systems that can be used to insert a transposon end into sample nucleic acids include, but are not limited to, those that use the EZ-Tn5™ hyperactive Tn5 Transposase available from EPICENTRE Technologies, Madison, Wis., or the Robust Tn5 Transposase available from Creative Biogene Biotechnology, Shirley, N.Y. USA.

Transposon end oligonucleotides that have the sequences of the corresponding transposon ends can be synthesized using an oligonucleotide synthesizer or purchased from a commercial source based on information available from the respective vendors or using information well known in the art. For example, the nucleotide sequences of the hyperactive transposon mosaic end for EZ-Tn5™ transposase are presented in U.S. Patent Publication No. 2010/0120098 (which is hereby incorporated by reference for its description of transposition systems) and additional information related to EZ-Tn5™ transposase is available in the published literature and online at www.EpiBio.com from EPICENTRE Biotechnologies, Madison, Wis., USA.

Transposition reactions can be carried out in any suitable reaction vessel, such as, for example, in microfuge tubes, wells of a microtiter plate or in compartments of a microfluidic device, such as those described below.

An illustrative in vitro transposition reaction in which a double-stranded transposon inserts into double-stranded target DNA is described in U.S. Pat. No. 10,526,601. First, a loaded transposase attacks the target DNA by making two staggered nicks on the opposite strands of the DNA. The distance between two nicks is transposase dependent. For example, it is 9 bases for Tn5. Then, the same loaded transposase links the 3′ end of transposon DNA to the 5′ end of target DNA, leaving a gap of 9 bases for Tn5 on the other strand at each joint. In some embodiments, this gap is filled and sealed using molecular biological techniques. For example, the gap can be filled by polymerase using dNTPs, and sealed by ligase. These two gaps are two identical repeated sequences, which can be used as barcodes.

### Transposons

Transposons useful in methods described here include wild-type Tn5 transposases and functional mutants or modifications thereof. The transposon ends are double-stranded, but the linking sequence need not be double-stranded.

In some embodiments, the transposon comprises identical transposon ends, for example, the two transposon ends have or comprise an identical nucleic acid sequence. In some embodiments, the transposon comprises identical terminal inverted repeat sequences. In some embodiments, the transposon comprises different transposon ends, where each end has or comprises a different nucleic acid sequence.

In some embodiments, the transposon includes at least one primer binding site. The primer binding site can be any nucleotide sequence to which a primer can anneal for the purpose of priming nucleotide polymerization. The primer binding site can be located in the stuffer sequence, e.g., adjacent to the transposon end or located within one of the transposon ends. In some embodiments, both transposon ends comprise the same primer binding site. The length of the primer binding site typically ranges from about 6 to about 50 nucleotides or more, e.g., about: 6, 7, 8, 9, 10, 11, 12, 13, 14, 15, 16, 17, 18, 19, 20, 25, 30 35, 40, 45, or 50 nucleotides, or any range defined by any of these values, e.g., 10-30 or 15-25 nucleotides.

In some embodiments, the primer binding site is used to amplify a target nucleic acid or a portion thereof. In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally. In some embodiments, the primer binding site is used to amplify a library of genomic fragments produced by tagmentation of genomic DNA. In some embodiments, the amplification is uniform and synchronized.

In some embodiments, the primer binding site is used for priming WGA. Each transposon need only have one such WGA primer site because transposition can be carried out so as to incorporate transposons, on average, so that they are close enough (e.g., about 500 bp to 300 Kb) to permit priming in adjacent transposons to amplify the intervening regions of the sample nucleic acids (e.g., genomic DNA). A primer binding site that is suitable for WGA will, in some embodiments, have a sequence that is either not found or present at low copy number in the sample nucleic acids so that priming occurs primarily at the first primer binding site.

To facilitate analysis of the nucleic acids produced upon transposition, optionally followed by WGA, one or more additional primer binding sites can be included in the transposons. Such additional primer site(s) can include, e.g., those that are suitable for amplifying the nucleic acids and/or subjecting them to DNA sequencing. In particular embodiments, these primer(s) can be located so that barcode sequence(s) and index sequence(s), if present, are amplified and/or sequenced together with their associated nucleic acid segment (i.e., the segment of sample nucleic acids adjacent to the location of transposon insertion).

In some embodiments, it will be advantageous to include a third primer binding site in each transposon. In some embodiments, the second and third primer binding sites are the same; in other embodiments, the second and third primer binding sites are different.

Representative examples of primer binding sites are described in U.S. Pat. No. 10,526,601, which is incorporated by reference herein.

In some embodiments, the transposon comprises a barcode sequence, as described below. In some embodiments, the transposon comprises a stuffer sequence.

In some embodiments, the transposon barcode sequence is located in the stuffer sequence, e.g., adjacent to the transposon end. In some embodiments, the transposon barcode sequence is located within one of the transposon ends, as described in more detail below. If desired, each transposon can include a second transposon barcode sequence, which can be the same as, or different from, the first transposon barcode sequence. The second transposon barcode sequence can be located in the stuffer sequence, e.g., adjacent to the transposon end or located within one of the transposon ends. For example, the second transposon barcode sequence can be located within or adjacent to a transposon end, with the first transposon barcode sequence is located within or adjacent to the other transposon end.

Transposon barcode sequences will have a length sufficient to encode the desired number of different barcodes. For example, if the barcode sequence includes three nucleotides, the number of possible different barcodes is 4³=64. Illustrative barcode sequence lengths are: 2, 3, 4, 5, 6, 7, 8, 9, 10, 11, 12, 13, 14, 15, 16, 17, 18, 19, 20, 25, 30 35, 40, 45, 50 nucleotides or more, and can fall within any range bounded by any of these values, e.g., 10-15 nucleotides. Barcode sequences can, but need not, be contiguous. Thus, for example, a barcode sequence may be characterized by two adjacent nucleotides, with a third nucleotide separated by a few intervening non-barcode nucleotides. Non-contiguous barcode sequence can, for example, be used for barcodes located within transposon ends (see below).

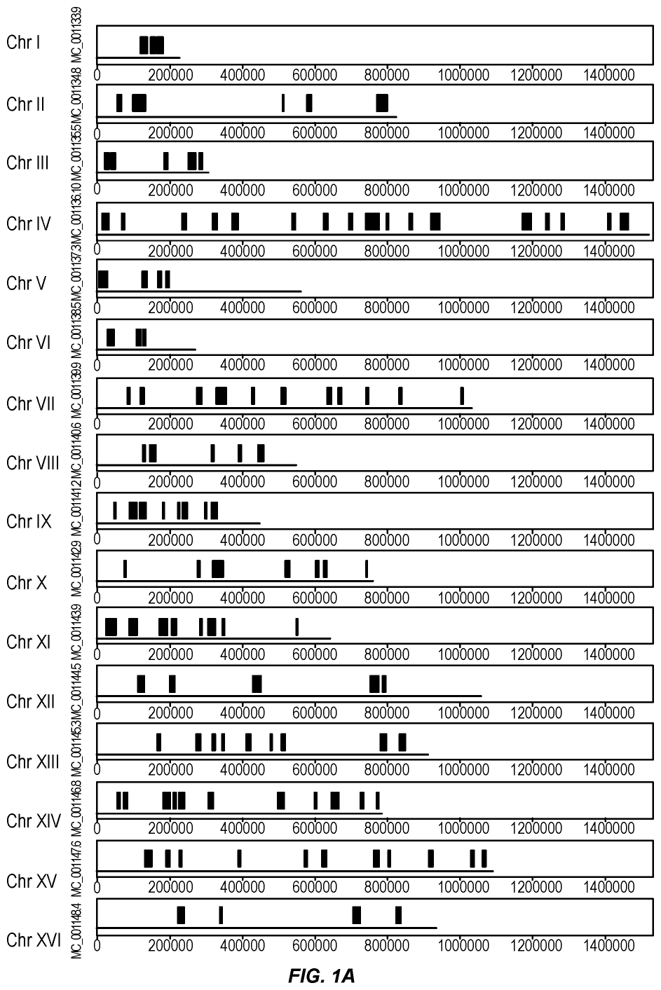

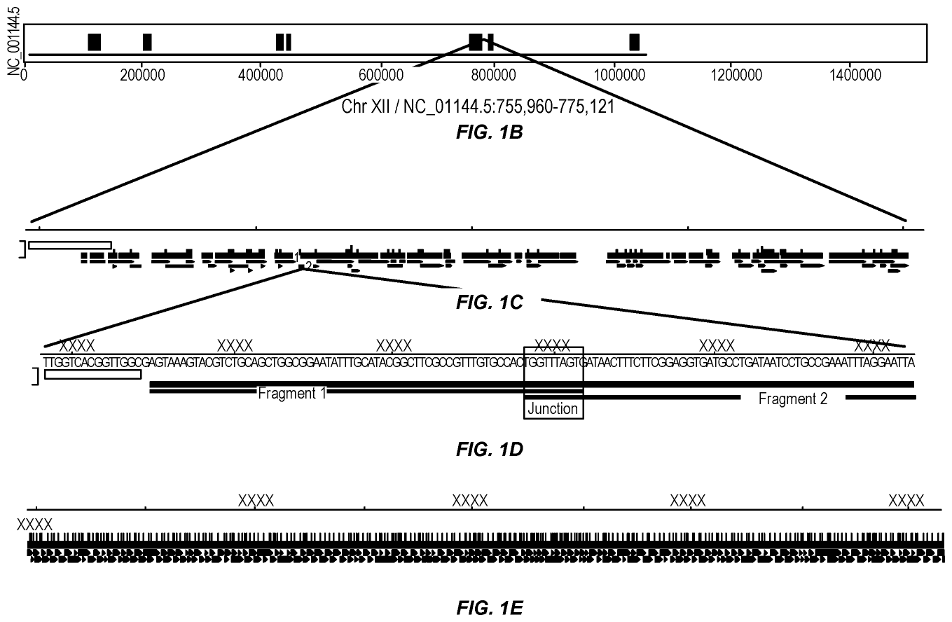

In particular embodiments, it may be advantageous to include one or more other types of barcodes/index sequences in the transposon. Such other sequences are termed “index sequences” herein, simply to distinguish them from the transposon barcode sequences discussed herein. Index sequences can be used, for example, to encode any desired kind of information regarding the barcoded nucleic acid molecules, such as the cell or cells or the reaction, from which the barcoded nucleic acid molecules were derived. If desired, each transposon can include a second index sequence, which can be the same as, or different from, the first index sequence. For example, one index sequence could be used to identify the cell from which the nucleic acids were derived, and the other could be used to identify a particular reaction to which they were subjected (e.g., a particular type of WGA). In an illustrative embodiment, pictured in FIG. 1, a transposon can include a first transposon barcode sequence located within or adjacent to one transposon end, and a second transposon barcode sequence located within or adjacent to the other transposon end, wherein the first index sequence is adjacent to the first barcode sequence, and the second index sequence is adjacent to the second barcode sequence.

The statements above regarding barcodes also apply to index sequences, which can be located in the stuffer sequence, e.g., adjacent to the transposon end or located within one of the transposon ends. In certain embodiments, a first index sequence is close enough to the first barcode sequence to ensure that both sequences will be included in one sequencing read. For example, one of these sequences can be located in the transposon end and one in the stuffer sequence adjacent thereto, or both sequences can be located in the transposon end or in the stuffer sequence, and/or the index sequence can be immediately adjacent to the barcode sequence. Index sequences can be any suitable length and contiguous or non-contiguous. Illustrative barcode sequence lengths are: 2, 3, 4, 5, 6, 7, 8, 9, 10, 11, 12, 13, 14, 15, 16, 17, 18, 19, 20, 25, 30 35, 40, 45, 50 nucleotides or more, and can fall within any range bounded by any of these values, e.g., 10-15 nucleotides.

Sets of transposons useful, e.g., in analyzing nucleic acids from multiple separate cells can be provided in a kit (see below). Such a kit can include two or more sets transposons, one for each cell to be analyzed. Each transposon within a set includes a different first transposon barcode sequence, and each set of transposons is characterized by a different index sequence, which be used to identify the cell under analysis.

In certain embodiments, the transposon ends are double-stranded. In such embodiments, the stuffer sequence can be double-stranded, discontinuous, or single-stranded, optionally with a 3′-3′ connection or 5′-5′ connection. The stuffer sequence should be sufficiently long to form a loop when the transposon ends are complexed with a suitable transposase. Because single-stranded DNA is more flexible than double-stranded DNA, a single-stranded stuffer sequence can be considerably shorter (e.g., about 50 nucleotides) than double-stranded stuffer sequence (e.g., about 500 nucleotides). Thus, illustrative stuffer sequences can range from about 45 to about 1000 nucleotides or more, e.g., about: 45, 50, 55, 60, 65, 70, 75, 80, 85, 90, 95, 100, 125, 150, 175, 200, 225, 250, 275, 300, 325, 350, 375, 400, 425, 450, 475, 500, 600, 650, 700, 725, 750, 800, 850, 900, 950, 1000, or can fall within any range bounded by any of these values, e.g. 50-550 nucleotides.

The transposon ends typically include the nucleotide sequences (the “transposon end sequences”) that are necessary to form the complex with a transposase or integrase enzyme that is functional in an in vitro transposition reaction. A transposon end forms a “complex” or a “synaptic complex” or a “transposome complex” (also referred to as a “transpososome”) with a transposase or integrase that recognizes and binds to the transposon end, and which complex is capable of inserting or transposing the transposon end into target DNA with which it is incubated in an in vitro transposition reaction. A double-stranded transposon end exhibits two complementary sequences consisting of a “transferred transposon end sequence” or “transferred strand” and a “non-transferred transposon end sequence,” or “non-transferred strand.” For example, one transposon end that forms a complex with a hyperactive Tn5 transposase (e.g., EZ-Tn5™ Transposase, EPICENTRE Biotechnologies, Madison, Wis., USA) that is active in an in vitro transposition reaction comprises a transferred strand that has a “transferred transposon end sequence” as follows: 5′ AGATGTGTATAAGAGACAG 3′ (SEQ ID NO:19) and a non-transferred strand that has a “non-transferred transposon end sequence” as follows: 5′ CTGTCT CTTATACACATCT 3′ (SEQ ID NO:20). In some embodiments, the transposon ends form a complex with Tn5 transposase (e.g., Robust Tn5 Transposase, Cat. No. EMQZ1422, Creative Biogene Biotechnology, Shirley, N.Y., USA) that is loaded with a single duplex formed by

NEX8a
(SEQ ID NO: 14)
(5′-CAGAGATGTGTATAAGAGACAG-3′)
and
Tn5Up
(SEQ ID NO: 15)
(5′- Phos-CTGTCTCTTATACACATCT-3′).

Different transposases utilize transposon ends that differ in length and sequence. For example, the Tn5 end requires about 17 nucleotides. Although, the length appears to be important for function, the base composition will tolerate some variations (Goldhaber-Gordon, et. al. J. Biol. Chem. 2002, 277:7703-7712, which is hereby incorporated by reference for its description of variable positions).

### Reconstruction of Singe Cell Genomes

The methods described herein can be used to reconstruct the genome of a single cell or a population of closely related cells, such as a clonal cell line. The methods can comprise disrupting a single cell to obtain genomic DNA from the cell, followed by tagmentation of the genomic DNA. The tagmentation step can be performed in a reaction mixture comprising a Tn5 transposase loaded with transposon ends (not a complete transposon) and the genomic DNA under conditions that result in fragmentation of the genomic DNA. Suitable conditions are described in the Examples. The transposase can be loaded with two identical transposon ends, and tagmentation produces a plurality of genomic fragments, where one or more, or a plurality of fragments are labeled with an identical transposon end at the 5′ and 3′ ends of the fragments. Thus, tagmentation can be used to produce a library of labeled genomic fragments.

After the tagmentation step, the transposase can be removed from the reaction mixture, for example, by digestion with a protease. The protease can be denatured to prevent digestion of enzymes added in downstream steps. The library of genomic DNA fragments can then be amplified for a low number of cycles (e.g., one, two, three, four, five, six, seven, eight, nine or ten cycles) using universal primers that hybridize to primer binding sequences in the transposon ends. The amplified fragments can be further amplified with external sequencing primers and the amplified fragments sequenced as described in the Examples.

To reconstruct the genome, the ends of the genomic fragments are then joined or linked together based on overlapping 8-10 nucleotide genomic sequence immediately next to the transposon end to restore the order of the fragments as originally present in the genome. The unique overlapping sequence is typically located 3′ or downstream of the transposon end. For example, the genomic fragments can be linked together in the original 5′ to 3′ order on the chromosome by concatenating neighboring fragments at the 8-10 nucleotide overlapping ends (transposon junctions).

As noted above and described in FIG. 3, some genomic fragments cannot be matched with a neighboring fragment based on defective cutting of the DNA by one reaction center of the transpososome. However, the genome can be accurately reconstructed by disregarding fragments that result from a defective Tn5 reaction.

In some embodiments, genomic DNA molecules are reconstructed by combining neighboring fragments through junctions to form a larger contiguous sequence (“contig”), also referred to as “islands.” In some embodiments, islands and fragments are assigned to each molecule, a process referred to as phasing, by following the rules of exclusivity and greediness. Exclusivity requires that any fragment is allowed to belong to only one molecule, and two overlapping fragments must belong to separate molecules unless they share a junction of 8-, 9-, or 10-bases. Greediness assigns as many islands and fragments as possible to the first molecule, then to the second, then the third, etc., until all islands are exhausted. Thus, the methods can determine the genomic sample's ploidy and assign each fragment to the correct DNA molecule.

### Methods for Counting DNA Molecules

In another aspect, the disclosure provides methods for accurately counting the number of DNA molecules in a sample. In some embodiments, the methods comprise similar steps as those for reconstructing the genome described above, where reconstructing the genome allows the number of separate, single molecules to be determined and counted as integers. In some embodiments, the method comprises (i) obtaining genomic DNA derived from a single fully disrupted cell; (ii) contacting and fragmenting the genome using a transposase loaded with two identical transposon ends to form a plurality of genomic fragments each labeled with an identical transposon end at its 5′ and 3′ ends; (iii) extending a complementary strand of each fragment using a universal primer comprising a sequence complementary to the identical transposon end to generate one or more extension products; (iv) determining the nucleotide sequence the extension products; (v) detecting one or more shorter extension products and one longer extension product comprising one identical segment of genomic sequence; (vi) disregarding the one or more shorter extension products; and (vii) identifying appropriate connections among the remaining extension products based on overlapping unique 8-10 nucleotide sequence immediately next to the transposon end to create a contiguous sequence; and (viii) assigning the contiguous sequence to a first or second DNA molecule, thereby counting or determining the number of individual DNA molecules.

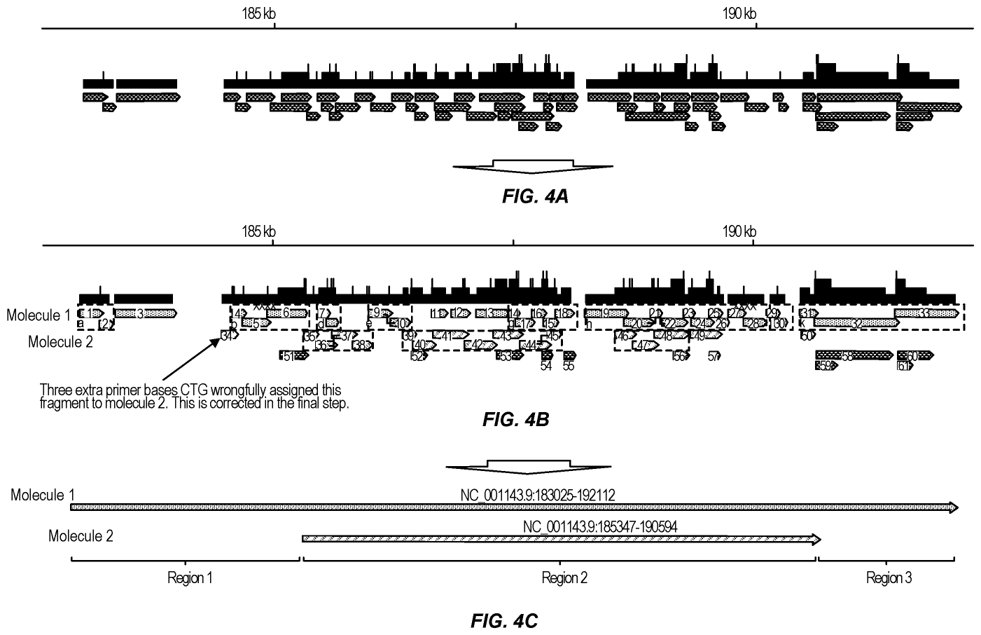

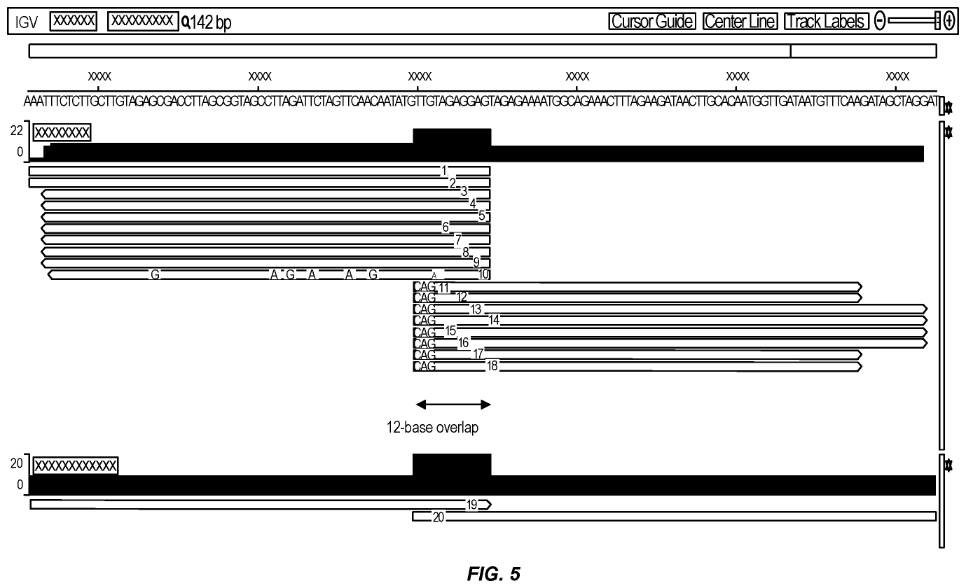

In some embodiments, the assigning step occurs using the rules of exclusivity and greediness. In some embodiments, an algorithm is used to re-assemble unique DNA molecules over a given region of the genome. In some embodiments, the algorithm starts from a fragment mapped to the very 5′ end of the reference genome and scans in the 3′ direction looking for an adjacent fragment that shares a transposon junction (e.g., a unique 8, 9 or 10 bp sequence) with the first fragment. Once the second fragment is identified, the algorithm semantically joins two fragments to form a contiguous structure referred to as an “island”. The greediness approach attempts to extend the length of the island as long as there are adjacent fragments that share transposon junction. In the case where a third mapped fragment overlapped with the first island, but cannot be joined with the island through a transposon junction, the exclusivity rule would assign the third fragment to a new molecule. Then, the algorithm would continue to the next fragment downstream and apply greediness rule to the existing DNA molecules by packing fragments and islands wherever they were logically possible. An example of the method is shown in FIG. 4. An exemplary embodiment is described in the examples.

In some embodiments, counting DNA molecules comprises counting digital DNA molecules.

### Methods for Sequencing Polynucleotides

In some embodiments, the nucleic acid molecules are analyzed, optionally after amplification, to determine the polynucleotide sequences of the genomic fragments associated with a given polynucleotide segment. Any available method capable of making this determination can be employed. In some embodiments, “next-generation” or “third generation” DNA sequencing is used to determine the polynucleotide sequence.

Next-generation sequencing techniques parallelize the sequencing process, producing thousands or millions of sequences concurrently. Illustrative next-generation techniques include, but are not limited to, Massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, and Heliscope single molecule sequencing.

Many next-generation sequencing techniques include an amplification step prior to DNA sequencing. For example, emulsion amplification or bridge amplification can be carried out. Emulsion PCR (emPCR) isolates individual DNA molecules along with primer-coated beads in aqueous droplets within an oil phase. PCR produces copies of the DNA molecule, which bind to primers on the bead, followed by immobilization for later sequencing. emPCR is used in the methods by Marguilis et al. (commercialized by 454 Life Sciences, Branford, Conn.), Shendure and Porreca et al. (referred to herein as “454 sequencing;” also known as “polony sequencing”) and SOLiD sequencing, (Life Technologies, Foster City, Calif.). See M. Margulies, et al. (2005) “Genome sequencing in microfabricated high-density picolitre reactors” Nature 437: 376-380; J. Shendure, et al. (2005) “Accurate Multiplex Polony Sequencing of an Evolved Bacterial Genome” Science 309 (5741): 1728-1732. In vitro clonal amplification can also be carried out by “bridge PCR,” where fragments are amplified upon primers attached to a solid surface. Braslaysky et al. developed a single-molecule method (commercialized by Helicos Biosciences Corp., Cambridge, Mass.) that omits this amplification step, directly fixing DNA molecules to a surface. I. Braslaysky, et al. (2003) “Sequence information can be obtained from single DNA molecules” Proceedings of the National Academy of Sciences of the United States of America 100: 3960-3964.

DNA molecules that are physically bound to a surface can be sequenced in parallel. “Sequencing by synthesis,” like dye-termination electrophoretic sequencing, uses a DNA polymerase to determine the base sequence. “Pyrosequencing” uses DNA polymerization, adding one nucleotide at a time and detecting and quantifying the number of nucleotides added to a given location through the light emitted by the release of attached pyrophosphates (commercialized by 454 Life Sciences, Branford, Conn.). See M. Ronaghi, et al. (1996). “Real-time DNA sequencing using detection of pyrophosphate release” Analytical Biochemistry 242: 84-89. Reversible terminator methods (commercialized by Illumina, Inc., San Diego, Calif. and Helicos Biosciences Corp., Cambridge, Mass.) use reversible versions of dye-terminators, adding one nucleotide at a time, and detecting fluorescence at each position in real time, by repeated removal of the blocking group to allow polymerization of another nucleotide.

In one embodiment of the detection-by-primer extension method, which can conveniently be carried out on the 454 sequencing platform, the first and second primer extension reactions are carried out sequentially in at least two cycles of primer extension. In particular, a first cycle of primer extension is carried out using the first primer that anneals to the first nucleotide tag, and a second cycle of primer extension is carried out using the second primer that anneals to the second nucleotide tag. All deoxynucleoside triphosphates (dNTPs) are provided in each cycle of primer extension. The incorporation of any dNTP into a DNA molecule produces a detectable signal. The signal detected in the first cycle indicates the presence of the first target nucleic acid in the nucleic acid sample, whereas the signal detected in the second cycle indicates the presence of the second target nucleic acid in the nucleic acid sample. Thus, each target nucleic acid (e.g., mutation) can be detected with only a single cycle of the sequencing platform.

So-called “third-generation” sequencing techniques aim to increase throughput and decrease the time to result and cost by reading sequence directly from single DNA molecules, thus eliminating the need for template amplification as in the case of bridge PCR or emulsion PCR. Illustrative third-generation techniques include Nanopore DNA sequencing, Tunneling currents DNA sequencing, Sequencing by hybridization, Sequencing with mass spectrometry, Microfluidic Sanger sequencing, Microscopy-based techniques, RNA polymerase (RNAP) sequencing, In vitro virus high-throughput sequencing.

In some embodiments, the sequences of the genomic DNA fragments are mapped to a reference genome. Methods for mapping sequences to a reference genome include, but are not limited to, the BOWTIE2 aligner (17) as described in the Examples. In some embodiments, the UFI sequences are used to map the individual genomic fragments to the reference genome.

### Transposon-Mediated Barcoding

Transposon-mediated barcoding may be used in any application in which the resultant transposon barcodes can be exploited in further analysis of the barcoded nucleic acid molecules. For a variety of genome-wide analyses, conditions are adjusted so that it is extremely unlikely that the same pattern of barcodes will appear in any two alleles. For example, ten different transposon barcodes (e.g., BC1, BC2, BC3, . . . BC10) can be employed in a reaction that inserts one transposon, on average, every 1000 bp. A 10 Kb region will, on average, contain ten transposons, with 10¹⁰ possible permutations in arrangement of transposons in this region. The insertion sites are substantially random because, although hot spots exist, they will be “filled in” by transposons during transposition, leaving the remaining transposons free to insert randomly. Assuming substantially random insertion, the total possible number of patterns of transposon barcodes is enormous, ensuring that the odds of two alleles for this region having the same barcodes incorporated into the same sites are vanishingly small. As long as this is true, the number of different barcode-nucleic acid segment combinations that include all or a portion of a locus of interest can be detected to determine the copy number of that locus. The detection of one such combination indicates that the locus is either haploid (e.g., a locus on the Y chromosome) or possible that allele-dropout has occurred in the course of the analysis. The detection of two such different combinations indicates that the locus is diploid. A number of types of differences are commonly observed in the case of a diploid locus and are described in U.S. Pat. No. 10,526,601.

Although most normal somatic human cells are diploid, consisting of 22 pairs of autosome and two sex chromosomes, some normal cells exist in polyploid forms. For example, cardiomyocytes are typically tetraploid, as are hepatocytes, while trophoblast cells in embryos have 1000 sets of chromosomes. In addition, some cancer cells have varying numbers of chromosomes, from less than 46 to more than 92. HeLa cells from one cell line at one time point, for example, have a total of 76 to 82 chromosomes. Among them, one cell has six copies of chromosome 5 and five copies chromosome 9 in a karyotyping study. Such cells are also characterized by lot of chromosomal translocation leading to mosaic structure. Other phenomena have been reported that lead to more than two sets of chromosomes in a single cell. For example, cell-in-cell formation arises from entosis. The methods described here can be used in characterizing all such deviations from the typical diploid situation.

In some embodiments, transposon-mediated barcoding is employed to identify one or more gains in copy number. More specifically, when the detected number of barcode-nucleic acid segment combinations is greater than the expected normal number of alleles for the locus, the sample is identified as one wherein the locus is at a higher-than-expected copy number in the cell. Multiple loci can be analyzed to distinguish between the gain of a particular locus, chromosomal sub-region, chromosome arm, or entire chromosome and a cell that has altered ploidy, e.g., a cell that is tetraploid, rather than diploid. For example, a high-resolution genome-wide analysis can be used to identify small gains throughout the genome, whereas low-resolution genome-wide analysis can be used to identify larger gains, e.g., of chromosome arms or entire chromosomes.

### Sample Nucleic Acids

Preparations of sample nucleic acids can be obtained from any source. The sample nucleic acids need not be in pure form but are typically sufficiently pure to allow the reactions of interest to be performed.

In particular, nucleic acids useful in the methods described herein can be extracted and/or amplified from any source, including bacteria, protozoa, fungi, viruses, organelles, as well higher organisms such as plants or animals, particularly mammals, and more particularly humans. Nucleic acids can be extracted or amplified from cells, bodily fluids (e.g., blood, a blood fraction, urine, etc.), or tissue samples by any of a variety of standard techniques. Illustrative samples include samples of plasma, serum, spinal fluid, lymph fluid, peritoneal fluid, pleural fluid, oral fluid, and external sections of the skin; samples from the respiratory, intestinal genital, and urinary tracts; samples of tears, saliva, blood cells, stem cells, or tumors. For example, samples of fetal DNA can be obtained from an embryo or from maternal blood. Samples can be obtained from live or dead organisms or from in vitro cultures. Illustrative samples can include single cells, formalin-fixed and/or paraffin-embedded tissue samples, and needle biopsies.

In certain embodiments, the methods described herein are used in the context of analyzing single cells, and, in some embodiments, single-cell analysis is carried out in a population of cells. In some embodiments, individual chromosomes from single cells can be isolated and analyzed.

Single-cell analysis can be carried out using any method whereby the nucleic acids of a single cell can be subjected to transposon-mediated fragmentation separately from any other cell, i.e., at/in a reaction site that is sufficiently separate from the reaction site for any other cell. In some embodiments, single-cell analysis entails capturing cells of a population in separate reaction volumes to produce a plurality of separate reaction volumes containing only one cell each. Cell-containing separate reaction volumes can be formed in droplets, in emulsions, in vessels, in wells of a microtiter plate, or in compartments of a matrix-type microfluidic device. In illustrative embodiments, the separate reaction volumes are present within individual compartments of a microfluidic device, such as, for example, any of those described in U.S. Patent Publication No. 2013/0323732, published May 12, 2013, Anderson et al. (hereby incorporated by reference for their descriptions of single-cell analysis methods and systems). The C₁™ Single-Cell Auto Prep System available from Fluidigm Corporation (South San Francisco, Calif.) provides bench-top automation of the multiplexed isolation, lysis, and reactions on nucleic acids from single cells in an “integrated fluidic circuit (IFC)” or “chip” and is therefore well-suited for performing transposon-mediated barcoding of nucleic acids from single cells. In particular, the C₁ Single-Cell Auto Prep Array™ IFC is a matrix-type microfluidic device that facilitates capture and highly paralleled preparation of 96 individual cells. When used properly, each capture site within the chip captures one single cell. Sometimes, a site may capture zero, two, or more cells; however, the exact number of captured cells in each captured site of a C.sub.1 chip is easily verified at high confidence and easily documented in a microscopic picture. In certain embodiments, cells are captured and transposon-mediated fragmentation is carried out in each separate reaction volume to produce barcoded nucleic acid molecules, which are analyzed, most conveniently by DNA sequencing, be it Sanger sequencing, next-generation sequencing, or third-generation sequencing, optionally after WGA.

In some embodiments, tagmentation and/or any subsequent steps, such as WGA or other amplification, is carried out in a microfluidic device having reaction chambers ranging from about 2 nL to about 500 nL. The lower the reaction chamber volume, the higher the effective concentration of any target nucleic acid and the greater the number of individual assays that may be run (either using different probe and primer sets or as replicates of the same probe and primer sets or any permutation of numbers of replicates and numbers of different assays).

In some embodiments, the analysis of the nucleic acids can be carried out in the same reaction volumes in which transposon-mediated fragmentation is carried out. In particular embodiments, however, it is advantageous to recover the contents of the separate reaction volumes after tagmentation for subsequent analysis. For example, if a nucleic acid amplification is carried out in the separate reaction volumes, it may be desirable to recover the contents for subsequent analysis, e.g., by DNA sequencing. The contents of the separate reaction volumes may be analyzed separately and the results associated with the cells present in the original reaction volumes. In embodiments, in which separate reaction volumes may contain more than one cell, single-cell analysis can be achieved by identifying reaction volume(s) containing only a single cell and only analyzing the contents of those reaction volumes.

In particular embodiments, the cell/reaction volume identity can be encoded in the reaction product using one or more (e.g., a combination of) transposon indexes, for example, as discussed above. Cell/reaction indexes can then be determined together with their linked barcoded nucleic acid molecules to associate these molecules with the cell/reaction volume from which they were derived. In certain embodiments, sets of separate reaction volumes are encoded, such that each reaction volume within the set is uniquely identifiable, and then pooled, with each pool then being analyzed separately from any other pool. Where single cell analysis is desired, but reaction volumes may contain more than a single cell, such embodiments may also entail determining which reaction volume(s) contained only a single cell. Because the corresponding cell/reaction index for each reaction volume is known, the results from the single-cell reaction volumes can be discriminated from the multi-cell reaction volumes.

The methods described herein can be used to analyze nucleic acids from any type of cells, e.g., any self-replicating, membrane-bounded biological entity or any non-replicating, membrane-bounded descendant thereof. Non-replicating descendants may be senescent cells, terminally differentiated cells, cell chimeras, serum-starved cells, infected cells, non-replicating mutants, anucleate cells, etc. Cells used in the methods described herein may have any origin, genetic background, state of health, state of fixation, membrane permeability, pretreatment, and/or population purity, among other characteristics. Suitable cells may be eukaryotic, prokaryotic, archaeon, etc., and may be from animals, plants, fungi, protists, bacteria, and/or the like. In illustrative embodiments, human cells are analyzed. Cells may be from any stage of organismal development, e.g., in the case of mammalian cells (e.g., human cells), embryonic, fetal, or adult cells may be analyzed. In certain embodiments, the cells are stem cells. Cells may be wildtype; natural, chemical, or viral mutants; engineered mutants (such as transgenics); and/or the like. In addition, cells may be growing, quiescent, senescent, transformed, and/or immortalized, among other states. Furthermore, cells may be a monoculture, generally derived as a clonal population from a single cell or a small set of very similar cells; may be presorted by any suitable mechanism, such as affinity binding, FACS, drug selection, etc.; and/or may be a mixed or heterogeneous population of distinct cell types. Cells may be disrupted, partially (e.g., permeabilized) to allow uptake of transposons or fully (e.g., lysed) to release interior components.

One advantage of the methods described herein is that they can be used to analyze virtually any number of single cells. In various embodiments, the number of single cells analyzed can be about 10, about 50, about 100, about 500, about 1000, about 2000, about 3000, about 4000, about 5000, about 6000, about 7,000, about 8000, about 9,000, about 10,000, about 15,000, about 20,000, about 25,000, about 30,000, about 35,000, about 40,000, about 45,000, about 50,000, about 75,000, or about 100,000 or more. In specific embodiments, the number of cells analyzed can fall within a range bounded by any two values listed above.

### Whole Genome Amplification

In some embodiments, nucleic acid molecules are subjected to a whole genome amplification (WGA) procedure to generate more DNA for subsequence analysis. Any available WGA procedure can be employed to amplify barcoded nucleic acid molecules. Suitable WGA procedures include, but are not limited to:

Primer extension PCR (PEP) and improved PEP (I-PEP)—PEP typically uses Taq polymerase and 15-base random primers that anneal at a low stringency temperature. The use of Taq polymerase implies that the maximal product length is about 3 kb.

Degenerated oligonucleotide primed PCR (DOP-PCR)—DOP-PCR is well-established, widely accepted, and technically straightforward method. DOP-PCR uses Taq polymerase and semi-degenerate oligonucleotides that bind at a low annealing temperature at approximately one million sites in the human genome. The first cycles are followed by a large number of cycles with a higher annealing temperature, allowing only for the amplification of the fragments that were tagged in the first step. DOP-PCR generates, like PEP, fragments that are in average 400-500 bp, with a maximum size of 3 kb, although a DOP-PCR method that was able to produce fragments up to 10 kb had been described.

Ligation-mediated PCR (LMP)—LMP uses endonuclease or chemical cleavage to fragment the genomic DNA sample and linkers and primers for its amplification. It was first described by Ludecke and coworkers and was later adapted for the WGA of small quantities of gDNA and single cells. Rubicon Genomics commercializes different kits (Omniplex) that allow for the amplification of RNA, DNA and methylated DNA sequences. Advantages include that the method is able to amplify degraded DNA and that all steps are performed in the same tube. A limitation is that it generates fragments only up to 2 kb.

T7-based linear amplification of DNA (TLAD)—TLAD is a variant on the protocol originally designed to amplify mRNA, which has been adapted for WGA. It uses Alu I restriction endonuclease digestion and a terminal transferase to add a polyT tail on the 3′ terminus. A primer is then used with a 5′ T7 promoter and a 3′ polyA tract, and Taq polymerase is used to synthesize the second strand. Then the sample is submitted to in vitro transcription reaction and posterior reverse transcription. A major advantage is that TLAD does not introduce sequence and length-dependent biases.

Multiple displacement amplification (MDA)—MDA is a non-PCR-based isothermal method based on the annealing of random hexamers to denatured DNA, followed by strand-displacement synthesis at constant temperature. It has been applied to small genomic DNA samples, leading to the synthesis of high molecular weight DNA with limited sequence representation bias. As DNA is synthesized by strand displacement, a gradually increasing number of priming events occur, forming a network of hyper-branched DNA structures. The reaction can be catalyzed by the Phi29 DNA polymerase or by the large fragment of the Bst DNA polymerase. The Phi29 DNA polymerase possesses a strand displacement activity and a proofreading activity resulting in error rates 100 times lower than the Taq polymerase.

The Rapisome™ pWGA (protein-primed WGA) is a whole genome amplification process marketed by BioHelix (BioHelix Corporation, A Quidel Company. 500 Cummings, Suite 5550. Beverly, Mass. 01915). Instead of using primers, the kit uses primase to synthesize primers on-site, generating multiple initiation sites for random, whole genome amplification.

Kits for WGA are available commercially from, e.g., Qiagen, Inc. (Valencia, Calif. USA), Sigma-Aldrich (Rubicon Genomics; e.g., Sigma GenomePlex® Single Cell Whole Genome Amplification Kit, PN WGA4-50RXN). The WGA step of the methods described herein can be carried out using any of the available kits according to the manufacturer's instructions.

In particular embodiments, the WGA step is limited WGA, i.e., WGA is stopped before a reaction plateau is reached. Typically, WGA is performed for more than two amplification cycles. In certain embodiments, WGA is performed for fewer than about 10 amplification cycles, e.g., between four and eight cycles, inclusive. However, WGA can be performed for 3, 4, 5, 6, 7, 8, or 9 cycles or for a number of cycles falling within a range defined by any of these values.

In embodiments in which a WGA primer binding site is included in the transposon, e.g., in the stuffer sequence or within a transposon end, WGA can be carried out using a primer that binds to this site. For many applications, it will be most convention to use transposons that all include the same primer binding site to facilitate WGA using just one primer. However, different transposons may carry different primer binding sites, if desired, in which case multiple corresponding primers can be employed in WGA. If multiple primers are employed, WGA can be carried out with all primers present in the reaction mixture or multiple separate reactions can be performed using different primers. When WGA is primed from a site in the transposon, the average transposon density should be sufficient that the particular WGA procedure used will proceed efficiently. In various embodiments, the values and ranges given above for barcode density define suitable transposon densities for WGA priming from a site in the transposon stuffer sequence.

In embodiments in which a primase recognition sequence is included in the transposon stuffer sequence, pWGA can be carried out using a primase that binds to these sites introduced to genome by transposition.

WGA can be carried out in the same reaction mixture as tagmentation or nucleic acid molecules can be recovered and then added to new WGA reaction mixture. When WGA is carried out in the same reaction mixture, in some embodiments, the transposase is inactivated, e.g., using EDTA and/or heat denaturation. In either case, WGA can be carried out using a microfluidic device, such as any of those described above.

### Kits Containing Transposons for Reconstructing Genomes

Also provided are kits that include one or more reagents useful for practicing one or more methods described herein. A kit generally includes a package with one or more containers holding the reagent(s), as one or more separate compositions or, optionally, as admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay. In specific embodiments, the kit includes one or more matrix-type microfluidic devices discussed above.

In particular embodiments, a kit can include a set of two or more functional transposons (i.e., that are each capable of being inserted into nucleic acids by a transposase). In some embodiments, the transposon ends are identical (i.e., comprise the same nucleic acid sequence). In some embodiments, the transposon ends comprise a primer binding site. In some embodiments, the transposon ends comprise the same primer binding site. In some embodiments, the transposon ends flank a stuffer sequence having a primer binding site. In certain embodiments, the transposons each include the same primer binding site in the stuffer sequence. In various embodiments, the number of transposons in the set is: 2, 3, 4, 5, 6, 7, 8, 9, 10, 15, 20, 25, 30, 35, 40, 45, or 50 or more. In some embodiments, one or more of the transposons further comprise a unique barcode sequence. In some embodiments, the number of transposons in a kit falls within a range bounded by any of these values, e.g., 5-25 or 10-15.

In certain embodiments, the kit includes at least two sets of two or more transposons, wherein each transposon within a set includes a barcode sequence that is different from all other barcodes in the set, but wherein each set of transposons includes the same set of barcode sequences as the other set(s) of transposons. Each transposon within a set can have an index sequence that is the same for all transposons within a set, but different than in the other set(s) of transposons. In various embodiments, each set can include any number of transposons, as described above, and the kit can include any number of sets, e.g., 2, 3, 4, 5, 6, 7, 8, 9, 10, 15, 20, 25, 30, 35, 40, 45, 50, 60, 70, 80, 90, 100 or more. In some embodiments, the number of transposon sets in a kit falls within a range bounded by any of these values, e.g., 10-100 or 40-50. In some embodiments, each set of transposons is provided a mixture in a single container.

Such kits can also, optionally, include one or more transposases capable of incorporating the transposons into sample nucleic acids. In some embodiments, the transposase(s) are packaged with their corresponding transposons. In particular embodiments, the transpose(s) are loaded with their corresponding transposons. In some embodiments, the kit includes a Tn5 transposase, or modified variant thereof having increased activity.

In certain embodiments, the kit additionally includes a primer that binds within the artificial transposon ends and primes polymerization of a nucleotide sequence, wherein a plurality of the different artificial transposon ends includes the same primer binding site. Such embodiments are useful, for example, in DNA sequencing. For sequencing, the primer binding site is preferably located so as to minimize the number of bases between the primer binding site and the template to be sequenced. In illustrative embodiments, the primer binding site is adjacent, or immediately adjacent (i.e., with no intervening bases), to any invariant transposon end nucleotide(s), which is/are adjacent, or immediately adjacent, to the template to be sequenced. In some embodiments, the primer binding site is adjacent, or immediately adjacent (i.e., with no intervening bases), to the end of the barcode sequence.

### Computer Devices and Systems

In some embodiments, the methods described herein can be wholly or partially implemented in the form of a set of instructions executed by one or more programmed computer processors such as a central processing unit (CPU) or microprocessor. Such processors may be incorporated in an apparatus, server, client or other computing device operated by, or in communication with, other components of a computer system. In some embodiments, a computer device and/or computer system is configured to implement one or more embodiments of a method of the present disclosure. The computer system can include additional subsystems such as a printer, a keyboard, a fixed disk, or a monitor, which is coupled to a display adapter. The additional subsystems can be interconnected by system bus. Peripherals and input/output (I/O) devices, which couple to an I/O controller, can be connected to the computer system by any number of means known in the art, such as a serial port. For example, the serial port or an external interface can be utilized to connect the computer device to further devices and/or systems, including a wide area network such as the Internet, a mouse input device, and/or a scanner. The interconnection via the system bus allows one or more processors to communicate with each subsystem and to control the execution of instructions that may be stored in a system memory and/or the fixed disk, as well as the exchange of information between subsystems. The system memory and/or the fixed disk may embody a tangible computer-readable medium.

One or more of the embodiments described herein can be implemented in the form of control logic using computer software in a modular or integrated manner. Alternatively, or in addition, embodiments may be implemented partially or entirely in hardware, for example, with one or more circuits such as electronic circuits, optical circuits, analog circuits, digital circuits, integrated circuits (“IC”, sometimes called a “chip”) including application-specific ICs (“ASICs”) and field-programmable gate arrays (“FPGAs”), and suitable combinations thereof. Based on the disclosure and teachings provided herein, a person of ordinary skill in the art will know and appreciate other ways and/or methods to implement one or more embodiments described herein using hardware and/or a combination of hardware and software.

Any of the software components, processes or functions described in this application may be implemented as software code to be executed by a processor using any suitable computer language such as, Python, Java, C++ or Perl using procedural or object-oriented programming paradigms. The software code may be stored as a series of instructions, or commands on a computer readable medium, such as a random access memory (RAM), a read only memory (ROM), a magnetic medium such as a hard-drive, a floppy disk or Universal Serial Bus (USB) drive, or an optical medium such as a CD-ROM. Any such computer readable medium may reside on or within a single computational apparatus, and may be present on or within different computational apparatuses within a system or network.

In some embodiments, the computer device and/or computer system is configured to implement an algorithm described herein. In some embodiments, the algorithm is a bioinformatics algorithm, such as the BIGseq algorithm. For example, the algorithm can reconstruct haploid DNA molecules by piecing together DNA fragments. In some embodiments, the algorithm can be used to identify two DNA molecules. In some embodiments, the algorithm starts from a fragment mapped to the very 5′ end of the reference genome and scans in the 3′ direction looking for an adjacent fragment that shares a transposon junction (e.g., a unique 8, 9 or 10 bp sequence) with the first fragment. Once the second fragment is identified, the algorithm semantically joins two fragments to form a contiguous structure referred to as an “island”. The greediness approach attempts to extend the length of the island as long as there are adjacent fragments that share a transposon junction. In the case where a third mapped fragment overlapped with the first island, but cannot be joined with the island through a transposon junction, the exclusivity rule would assign the third fragment to a new molecule. Then, the algorithm would continue to the next fragment downstream and apply greediness rule to the existing DNA molecules by packing fragments and islands wherever they were logically possible. An example of the method is shown in FIG. 4. An exemplary embodiment of the algorithm is described in the Examples.

In some embodiments, the algorithm can be used to eliminate artifacts that occur due to defective transposase digestion of one strand of a double stranded DNA molecule as described above.

### EXAMPLES

### Example 1

This example describes a representative method for reconstructing a single nucleic acid molecule using tagmentation to generate fragments and assembling the fragments into a single molecule by mapping and concatenating through junctions.

Methods

Construction of Tn5 Transpososome with Identical Ends.

A Tn5 transpososome was constructed with identical transposomes ends by loading transposase (Cat. No. EMQZ1422) from Creative Biogene, Shirley, N.Y., with a single duplex formed by NEX8a (5′-CAGAGATGTGTATAAGAGACAG-3′) (SEQ ID NO: 14) and Tn5Up (5′-Phos-CTGTCTCTTATACACATCT-3′) (SEQ ID NO: 15) according to the protocol provided by the manufacturer. This transpososome was let to sit at 4° C. overnight before it was used for library construction.

### Construction of Primary Library, Library Amplification, and Sequencing.

The constructed transpososome was used to tagment about 3 femtograms of yeast genomic DNA (ATCC Cat. No. 9763) to generate a primary library. The transposase was removed from DNA by Protease K (New England Biolabs, Cat. No. P8107S) treatment and then Protease K was denatured by heat. The primary library was sequentially amplified by Phusion Hot Start II High-Fidelity PCR Master Mix (Thermo Fisher Scientific™ Cat. No. F565L) with NEX8a for three cycles, and then together with Illumina Read1 (5′-TCGTCGGCAGCGTCAGATGTGTATAAGAGACAG-3′) (SEQ ID NO: 16) and Read2 (5′-GTCTCGTGGGCTCGGAGATGTGTATAAGAGACAG-3′) (SEQ ID NO: 17) primer pair for 20 cycles, both with 30-min extension time at 65° C. The amplified library was cleaned with AMPure® (Beckman Coulter Cat. No. A63882) beads twice and eluted in 20 μL 10 Tris buffer, 8 μL of the eluents was taken to be barcoded in a 20 μL reaction in 6 cycles. The barcoded libraries were cleaned with AMPure beads twice and loaded onto MiSeq™ (Illumina, Cat. No. MS-102-3001) for sequencing 80 bases for Read1 and 70 bases for Read2, after quantitation following the manufacturer's protocol.

### Adapter Trimming.

The MiSeq™ output files were first passed through the Illumina® primary analysis pipeline to remove the majority of the primer and adapter sequences. The resulting paired end FASTQ files were evaluated for the percentage of reads from yeast genome relative to other known genomes using FastQ Screen (see the internet at www.bioinformatics.babraham.ac.uk/projects/fastq screen/). This served as a check for yield and contamination. The same FASTQ files were sent through Trim Galore (see the internet at www.bioinformatics.babraham.ac.uk/projects/trim_galore/), which used Cutadapt (see the internet at cutadapt.readthedocs.io/en/stable/index.html) and FastQC see the internet at www.bioinformatics.babraham.ac.uk/projects/fastqc/) as software engines, for removal of any remaining Illumina Read 1 and Read 2 primer adapters. The Trim Galore stringency for adapter matching was set at 15 bp (default 1); minimum Phred score was set at 20 (default); and any read shorter than 25 bp after quality or adapter trimming was discarded. All other Trim Galore parameters were at their default values.

### Mapping.

The Trim Galore processed read sequences in FASTQ format were mapped to the reference yeast genome (GenBank assembly accession: GCA_000146045.2) using the BOWTIE2 aligner17 with default parameters except the maximum fragment length for valid paired-end alignment (−X option) was set at 1000. The aligner generated a raw BAM file. The in-house Python scripts reconstructed fragments in BAM format to represent an amplified library (Column 2 of Table 1, the number of reads is shown in Column 4). During this process, two reads would merge if the fragment length was shorter than or equal to 150 bases, while a gap between the two reads was filled in with the reference genome sequences when the fragment length was greater than the combined read lengths of 150 bases. The file was then deduplicated to remove PCR duplications to represent the primary library (Column 3 of Table 1, the number of reads is shown in Column 5).

### Reconstruction of DNA Molecules.

A web browser-based Integrative Genomics Viewer (IGV) was modified to display how various mapped fragments created during the tagmentation process can be re-assembled into their originating DNA molecules by concatenating them at their 9-base overlapping ends, also referred to as “junctions.” Specifically, the BIGseq algorithm used an exclusivity and greediness approach to re-assemble unique DNA molecules over a given region of the genome: the algorithm started from a fragment mapped to the very 5′ end of the reference genome and scanned towards the 3′ direction looking for an adjacent fragment that shares a 9-base junction with the first fragment. Once the second fragment was identified, the algorithm semantically joins two fragments to form a contiguous structure referred to as an “island”. The greediness approach would attempt to extend the length of the island as long as there is an adjacent fragment that shares a 9 base junction. In case a third mapped fragment that overlapped with the first island but cannot be joined with the island through a 9-base junction, the exclusivity rule would assign the third fragment to a new molecule. Then, the algorithm would continue to the next fragment downstream and apply greediness rule to the existing DNA molecules by packing fragments and islands wherever they were logically possible.

### The GC Preference of Tn5 and the Identification of Uncanonical Junctions.

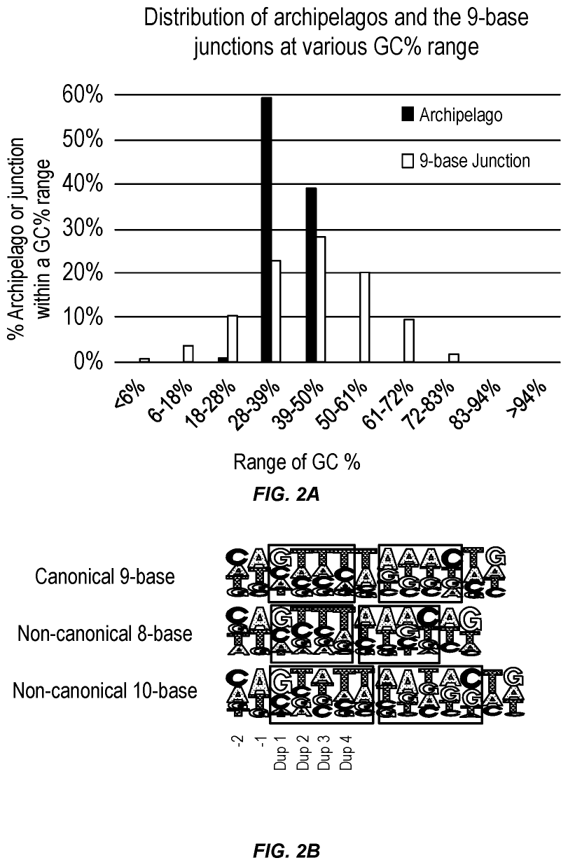

The transpososome was reported to show biases in choosing its cutting sites. If the bias is strong, it could be detrimental to the high coverage requirement of BIGseq. Incongruously, no GC bias was observed in the Nextera® libraries. Consistent with both sides of the earlier findings, the libraries described herein had similar GC profiles to that of the whole yeast reference genome. At the same time, the junctions showed a higher GC average than the average of the genome while exhibiting extreme broad GC distribution (FIG. 2A). The indiscriminating cutting by Tn5 ensured the high coverage of the libraries described herein. Tn5 transposase is a homodimer, exhibiting twofold axis of symmetry with each monomer containing one active center.

The transpososome preferred G immediately inside the cutting site (the Dup1 position), and A/T immediately outside the cutting site (−1); it slightly preferred at −2 position, while it is unscrupulous at the rest of the positions (FIG. 2B). Interestingly, in addition to 1889 9-base junctions in Sample 6Y5j, there were 97 8-base, and 139 10-base overlaps, respectively. The occurrence of 8- or 10-base overlaps was higher than the random chance. Ten-base junction has not been used to link fragments since it was reported 30 years ago.

### Base Preferences at Junctions.

Overlapping sequences with 8-, 9-, and 10-bases and their surrounding sequences were extracted and analyzed at the sequence logo site for the relative frequencies of every base at every position (see the internet at weblogo.berkeley.edu/logo.cgi).

The base preferences exhibited by both 10- and 8-base junctions around the cutting site are almost identical to those of the 9-base junctions (FIG. 2B). Therefore, it was concluded that Tn5 had at least two uncanonical cuts. Inclusion of uncanonical junctions helps credible reconstruction of the input molecules.

### Results

High coverage and specific mapping lead to the identification of archipelagos reflecting input DNA.

Integer counting of haploids in BIGseq is based on the deterministic property of tagmentation: a long stretch of double-stranded DNA can generate only one unique pattern upon tagmentation. The pattern can be determined bioinformatically as long as each fragment is mapped accurately and the junctions, 9-base overlaps between fragments created by transpososomes15,16, is identified correctly.

When mapping in-silico generated reads, 3% of these reads were mismapped by the BOWTIE2 aligner (17). Such mismapped reads typically landed to some specific locations of the genome. For comparison, a significantly higher percentage of reads were found to be mismapped by BWA-MEM aligner (18). Thus, BOWTIE 2 was selected for the studies described herein.

Table 1 summarizes the names, corresponding files, and key parameters of the actual yeast samples. The average lengths of fragments are slightly over 220 bases. The duplication rate, which is defined as the ratio between the total fragment number and the fragment number with unique fragment index (UFI)13, is between 3.1 and 7.4. The percentage of the genome covered by the fragments of each library ranges from 6.6% to 9.2%.

TABLE 1

| | | | Total | | | | |
|---|---|---|---|---|---|---|---|
| | | | fragments | Fragment | Average | | Percentage |
| | | | Mappable to | number with | duplication | Average | genome |
| Sample | | | yeast before | unique UFI | rate of | length of | covered by |
| name | Raw BAN file | Library-representing BAM file | Dedupe | after dedupe | fragments | fragments | fragments |
| 6Y5j | 6Y5j_S37_Ckite.bam | 6Y5j_S37_Ckite_pemerged_deduped.bam | 79,567 | 12,436 | 5.4 | 233.8 | 8.8% |
| 6Y5l | 6Y5l_S38_Ckite.bam | 6Y5l_S38_Ckite_pemerged_deduped.bam | 103,722 | 13,930 | 7.4 | 233.1 | 9.2% |
| 6Y5n | 6Y5n_S39_Ckite.bam | 6Y5n_S39_Ckite_pemerged_deduped.bam | 57,997 | 12,488 | 4.6 | 257.3 | 8.6% |
| 6Y5p | 6Y5p_S40_Ckite.bam | 6Y5p_S40_Ckite_pemerged_deduped.bam | 88,350 | 13,618 | 6.5 | 225.0 | 8.7% |
| 6Y6b | 6Y6b_S41_Ckite.bam | 6Y6b_S41_Ckite_pemerged_deduped.bam | 28,875 | 9,232 | 3.1 | 225.4 | 6.6% |
| 6Y6d | 6Y6d_S42_Ckite.bam | 6Y6d_S42_Ckite_pemerged_deduped.bam | 40,055 | 9,984 | 4.0 | 220.8 | 7.1% |
| 6Y6f | 6Y6f_S43_Ckite.bam | 6Y6f_S43_Ckite_pemerged_deduped.bam | 50,127 | 9,760 | 5.1 | 220.7 | 7.0% |
| 6Y6h | 6Y6h_S44_Ckite.bam | 6Y6h_S44_Ckite_pemerged_deduped.bam | 48,896 | 9,440 | 5.2 | 222.3 | 6.5% |

The sequencing files were visualized on the modified web browser-based IGV (19, 20). Most of the reads were in clusters, which are referred to as “archipelagos,” while nearly 90% of genome regions were almost blank (FIG. 1A), with scattered lonely fragments that consisted of 10% of total deduplicated fragments and covered less than 1% of the blank regions of the entire genome. All of the solo fragments were manually inspected. Most, if not all, were smaller than two hundred bases, and do not have a possible second mapping site. It was hypothesized that they were from low-level cross-contamination. Because the frequency of these fragments was very low and their appearance in all the samples was randomly distributed, their existence did not significantly affect the data processing on archipelagos.

FIG. 1A is a screenshot of a representative archipelagos, presented in blue bars, of sample 6Y5j of all chromosomes excluding mitochondrial DNA. 9.3% of genome was covered by 129 archipelagos, ranging from 1 Kb to 30 Kb, with a median size of 7.9 Kb and a median coverage of 79%. The average GC % of all archipelagos was indistinguishable from that of blank regions outside the archipelagos, and the average GC % of the library-covered regions was indistinguishable from that of uncovered regions within archipelagos, suggesting low GC bias in the library construction process. The high coverage rates within archipelagos foretell that there is a low chance that any archipelagos were missed. FIG. 1B-E depict a representative result of a library-representing file. The whole Chr XII/NC_001144.5 in BED format is displayed in Panel B, with nine archipelagos shown in blue bars. Among these nine archipelagos, a 19-Kb archipelago located in the region Chr XII/NC_001144.5) of 755,960-775,121 is zoomed in Panel C using a BAM file. This archipelago was made of a total of 75 fragments, with 36 junctions. One of the junctions is zoomed in Panel D. An in-silico mapping simulation of random tagmentation of a haploid covering the same region as Panel C is exhibited in Panel E. A perfect tiling pattern of this simulation is conspicuous, and it serves as an indicator of high specificity in mapping.

Improved Junction Identification by Trimming Remaining Primer Sequences.

Trim Galore (see the internet at github.com/FelixKrueger/TrimGalore) identified about 0.1% of the FASTQ reads output from the Illumina® primary processing pipeline still having remnant primer sequences and hence trimmed them. It also recognized that no more than 0.5% of the reads had Phred score less than 20 and they were discarded. Because the Trim Galore's stringency for adapter matching to a minimum of 15 bases to avoid false positives, it is possible that some small pieces of the primer sequence still remain attached to the reads. Indeed, 12 cases of missed junctions resulting from incomplete removal of primer sequences were identified. A case in point is the 12-base overlap presented at Chr XIV/NC_001146.8: 658,350-658,629. Of these 12 bases, only 9 bases truly overlap, while the three bases at the very 5′ end of the downstream fragment, CAG, are not shared with the upstream fragment, nor are they present in the reference genome (FIG. S1). This subsequence was identified to be the last three bases of primers used in PCR and it was removed to restore the real junction.

The GC Preference of Tn5 and the Identification of Uncanonical Junctions.

The transpososome was reported to show biases in choosing its cutting sites (refs. 21, 22). If the bias is strong, it could be detrimental to the high coverage requirement of BIGseq. Incongruously, no GC bias was observed in the Nextera® libraries23. Consistent with both sides of the earlier findings, the libraries had similar GC profiles to that of the whole yeast reference genome. At the same time, the junctions showed a higher GC average than the average of the genome while exhibiting extreme broad GC distribution (FIG. 2A). The indiscriminating cutting by Tn5 ensured the high coverage of the libraries.

Tn5 transposase is a homodimer, exhibiting twofold axis of symmetry with each monomer containing one active center16. The transpososome preferred G immediately inside the cutting site (the Dup1 position), and A/T immediately outside the cutting site (−1); it slightly preferred C at −2 position, while it is unscrupulous at the rest of the positions (FIG. 2B).

In addition to 1889 9-base junctions in Sample 6Y5j, there were 97 8-base, and 139 10-base overlaps, respectively. The occurrence of 8- or 10-base overlaps was higher than the random chance. Ten-base junction was reported more than 30 years ago (ref. 24) and has been rarely cited since. The base preferences exhibited by both 10- and 8-base junctions around the cutting site which was similar to those of the 9-base junctions (FIG. 2B) lead to the conclusion that Tn5 had at least two uncanonical cuts. Uncanonical cuts shed light on the reaction mechanisms of transpososomes and the inclusion of uncanonical junctions helps credible reconstruction of the input molecules by BIGseq as shown in the following sections.

Reconstruction of Mono-Ploidic Molecule and Identifications of Artifacts Caused by Defective Tn5.

The first step of the BIGseq pipeline to reconstruct DNA molecules is to chain neighboring fragments through junctions to form larger contigs, referred to as islands. Then islands and fragments are assigned to each molecule, a process referred as phasing, by following the rules of exclusivity and greediness. Exclusivity requires that any fragment is allowed to belong to only one molecule, and two overlapping fragments must belong to separate molecules unless they share a junction of 8-, 9-, or 10-base. Greediness asks to assign as many islands and fragments as possible to the first molecule, then to the second, then the third, etc., until all islands are exhausted. As only 10% of the genome was present in each sample, the majority of the archipelagos must have been mono-ploid, i.e., most of the archipelagos were made of only one molecule. A case in the point was the 4.8 Kb archipelago located at Chr V/NC_001137.3: 22,504-27,231 (FIG. 3A), this molecule was 82% covered by 20 fragments. The reconstruction process started with chaining 18 fragments into six non-overlapping islands (a-f), then assigning them to Molecule 1. The fragments in islands were depicted in a darker color and arranged alternatively between two lanes in the modified IGV. Lastly, the unchained Fragment 1, shown in a lighter tone, joined islands a-f to complete the reconstruction of Molecule 1. By the rule of exclusivity, Fragment 4 was temporarily set to be the lone candidate for the second molecule.

Fragment 4 had a 5′ end identical to Fragment 3, and both shared a 9-base junction with Fragment 2. Analyzing archipelagos of Sample 6Y5j revealed that quite a few fragments shared identical 5′ or 3′ ends. After examining the raw BAM files, it was determined that Fragment 4 was not a product of sequencing or alignment errors. Because the sequence within Fragment 3 shared no significant similarity to the sequences of the primers used in PCR or sequencing, it is unlikely that Fragment 4 was a PCR by-product resulting from mispriming on Fragment 3. While not being bound by theory, based on the shared junction, a novel hypothesis is that Fragment 4 resulted from an unreported defective Tn5 transpososome, of which one of the two reaction centers failed to make a nick to complete tagmentation. As Fragment 4 was identified as an artifact, this archipelago was reconstructed to be one molecule.

While not being bound by theory, a detailed mechanism is proposed in FIG. 3B to account for the observations: Transpososome made a single tagmentation at the bottom strand, a scenario denoted as “0/1”, where “0” represents the defective center and “1” represents the productive center of dimeric Tn5 transposase. The digits above and below “I” represent the top and the bottom strands, respectively. During the extension step, the strands that ligated to transposon end molecules—TE1, TE2, and TE3—will extend from 3′ end to become functional templates for the next round, eventually generating two indistinguishable long pieces and a short piece sharing the 5′ end. In a similar fashion, a I/O scenario will generate a short fragment sharing the 3′ ends with two indistinguishable longer fragments (FIG. 3C).

If the hypothesis holds true, it was expected that there will be four more complex patterns emerging when two defective transposomes react side by side, as described below:

Complex Scenarios 00/11 and 11/00, where two defective reaction centers are adjacent to one another on the top strand (FIG. 3D) or on the bottom 5 strand (FIG. 3E) respectively, resulting in four fragments (three unique sequences of varying lengths) sharing identical 5′ end or 3′ ends respectively.

Complex Scenario 01/10, where the first defective reaction center is upstream on the top strand and the second defective reaction center is downstream on the bottom strand, resulting in four fragments, two shorter fragments with one sharing 5′ end and the other sharing 3′ end with the other two identical longer fragments (FIG. 3F).

Complex Scenario 10/01, where the first defective reaction center is upstream on the bottom strand, and the second defective reaction center is downstream on the top strand, leading to two overlapping fragments (FIG. 3G).

As predicted, a significant number of these complex artifacts were identified. Some of the examples are shown in FIG. 6. These complex scenarios further reinforced the hypothesis. An algorithm to eliminate these artifacts was implemented, and the identified archipelago calls appeared to be cleaner and cohesive, as shown in the next section.

Complex Scenario 10/01 generates two overlapping fragments, a pattern that is similar to the result if they came from two separate molecules (FIG. 6).

It is worth pointing out that Fragment 8 and Fragment 9 (FIG. 3A) share an 8-base junction, a point discussed in a previous section. If this junction was not recognized, then Fragments 9, 10, 11, 12, and 13 would have had to be in Molecule 2, leaving a big gap in Molecule 1, an unlikely scenario under the experimental conditions.

Reconstruction and Counting of Two DNA Molecules by BIGseq.

The identification of the artifacts mentioned in the previous sections allowed the reconstruction of molecules in more complex situations as shown in FIG. 4. Panel A shows an archipelago identified in the region of Chr XI/NC_001143.9:183025-192112, with 61 deduplicated fragments. Among these 61 fragments, eleven fragments (Fragment 51-61) are sorted into the redundant group (maroon bars in Panel B) because each of them shares a common 5′ or 3′ end with a longer fragment. In a similar process described in a previous section, a total of eleven multi-fragment islands are then formed and marked in dashed boxes in FIG. 4B. Then, by following the rules of exclusivity and greediness, Islands a, b, d, e, g, h, j, and k are assigned to Molecule 1 (colored in orange), and Islands c, f, and j are assigned to Molecule 2 (colored in blue). Next, Fragments 3, 11, 12, and 13 are assigned to Molecule 1 and Fragment 50 joins Molecule 2.

The BIGseq algorithm further identified that Fragment 34 has three extra bases, CTG, at its 3′ end. These bases should be trimmed because they are the last three bases of the primer. Removing these three bases allows Fragment 34 to chain with Island b through a canonical junction. In the final step, Molecule 1 is set between 183,025 and 192,112 and Molecule 2 is set between 185,347 and 190,594, as shown in Panel C, to conclude the reconstruction of two homologous mono-ploid molecules. At the completion, Region 2 has two copies, Region 1 and Region 3 have one copy, and areas 3′ immediately adjacent to region 1 and 5′ to Region 3 have 0 copies (FIG. 4C).

Several points need to be stressed: a) it is unambiguous that Fragments 19-26 originated from one molecule, referred to as “in phase,” as they are chained by junctions (FIG. 4B). 2) The same is not necessarily true for Fragments 1-34 because there are gaps between Fragments 3 and 4, 18 and 19, 28 and 29, 30 and 31, 45 and 46, and 49 and 50. b) Although there are gaps between Fragments 8 and 9, 10 and 11, 11 and 12, 12 and 13, 13 and 14, 38 and 39, it is of high confidence that Fragment 34 and Fragments 4-18 originate from one haploid molecule while Fragments 35-45 originate from a different molecule. Although neither haploid molecule is contiguously covered, BIGseq is the first technology to enable physical connections to be established through the rule of exclusivity.

Single-cell DNA sequencing methods are often treated as bulk sequencing methods that are merely applied to single cell samples. The apparent similarity of the sample preparation and processing procedures shared by single cell and bulk sequencing protocols may reinforce this notion. For instance, similar to a standard Nextera bulk sequencing protocol (23), BIGseq also utilizes the following sample prep and processing procedures:

1) tagmentation of genomic DNA to construct the primary library,

2) amplification of the library,

3) sequencing the library using NGS,

4) mapping and deduplication and,

5) haploid analyses.

However, due to the heterogeneity of gene copy numbers in cancer cells and the undefined number of genomes in the bulk sample, the copy number per genome determined by Nextera-prepared bulk genomes may be calculated to be a decimal, a number that does not exist in nature and creates a conundrum to make sense at the biological level. However, copy numbers can be determined discretely when each of the genomes is interrogated individually.

Unfortunately, current single cell DNA sequencing technologies fail to capture the discreteness, as they adopt secondary analysis methodology developed for bulk sequencing (12). These statistics-heavy methodologies are not robust; the inferred copy number varies when a different bin size is chosen, or a different segmentation option is selected, or ploidy of the genome deviates from diploid (12). In contrast, as described herein, a different bioinformatic approach was used which is built upon the deterministic array of fragments generated by tagmenting a stretch of DNA. It simply pieces those fragments back into a single molecule by mapping and concatenating through junctions. Along with its simplicity, this approach carries with it the ability to count DNA molecules and identify mutations at single base resolution to specific haploids.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### REFERENCES

1. Ramamoorthy A, and Skaar T C. Gene copy number variations: it is important to determine which allele is affected. Pharmacogenomics 12, 299-301 (2011).
2. Sansregret L, Vanhaesebroeck B, and Swanton C. Determinants and clinical implications of chromosomal instability in cancer. Nat Rev Clin Oncol. 15, 139-150 (2018).
3. Almeida L V, Coqueiro-Dos-Santos A, Rodriguez-Luiz G F, McCulloch R, Bartholomeu D C, and Reis-Cunha J L. Chromosomal copy number variation analysis by next generation sequencing confirms ploidy stability in Trypanosoma brucei subspecies. Microb Genom. 4, e000223 (2018).
4. Gilchrist C, and Stelkens R. Aneuploidy in yeast: Segregation error or adaptation mechanism? Yeast 36, 525-539 (2019).
5. Ravichandran M C, Fink S, Clarke M N, Hofer F C, and Campbell C S. Genetic interactions between specific chromosome copy number alterations dictate complex aneuploidy patterns. Genes Dev. 32, 1485-1498 (2018).
6. Hirpara A, Bloomfield M, and Duesberg P. Speciation Theory of Carcinogenesis Explains Karyotypic Individuality and Long Latencies of Cancers. Genes (Basel) 9, 402 (2018).
7. Akao T. Progress in the genomics and genome-wide study of sake yeast. Biosci Biotechnol Biochem. 83, 1463-1472 (2019).
8 Levsky J M, and Singer R H. Fluorescence in situ hybridization: past, present and future. J Cell Sci. 116, 2833-2838 (2003).
9. Berisha S Z, Shetty S, Prior T W, and Mitchell A L. Cytogenetic and molecular diagnostic testing associated with prenatal and postnatal birth defects. Birth Defects Res. 112, 293-306 (2020).
10. Andriani G A, et al. A direct comparison of interphase FISH versus low-coverage single cell sequencing to detect aneuploidy reveals respective strengths and weaknesses. Sci Rep. 9, 10508 (2019).
11. Xi L. Single Cell DNA Sequencing—from Analog to Digital. Cancer Research Frontiers 3, 161-169 (2017). 12. Mallory X F, Edrisi M, Navin N, Nakhleh L Assessing the performance of methods for copy number aberration detection from single-cell DNA sequencing data. PLoS Comput Biol. 16, e1008012 (2020).
13. Xi L, et al. New library construction method for single-cell genomes. PLoS One, 12, e0181163 (2017).
14. Xi L, Leong, P, and Mihajlovic, A. Preparing Single-cell DNA Library Using Nextera for Detection of CNV. bio-protocol 9, e3175 (2019).
15. Berg D E, Schmandt M A, and Lowe J B. Specificity of transposon Tn5 insertion. Genetics 105, 813-828 (1983).
16. Davies D R, Goryshin I Y, Reznikoff W S, and Rayment I. Three-dimensional structure of the Tn5 synaptic complex transposition intermediate. Science 289, 77-85 (2000).
17 Langmead B, and Salzberg S L. Fast gapped-read alignment with Bowtie 2. Nat Methods 9, 357-359 (2012).
18 Li H. Aligning sequence reads, clone sequences and assembly contigs with BWA-MEM. arXiv. 1303, 3997 (2013).
19. Robinson J T, et al. Integrative genomics viewer. Nat Biotechnol. 29, 24-26 (2011).
20. Thorvaldsdottir H, Robinson J T, and Mesirov J P. Integrative Genomics Viewer (IGV): high-performance genomics data visualization and exploration. Brief Bioinform. 14, 178-192 (2013).
21. Lodge J K, Weston-Hafer K, and Berg D E. Transposon Tn5 target specificity: preference for insertion at G/C pairs. Genetics 120, 645-650 (1988).
22. Green B, Bouchier C, Fairhead C, Craig N L, and Cormack B P. Insertion site preference of Mu, Tn5, and Tn7 transposons. Mob DNA 3, 3 (2012).
23. Adey A, et al. Rapid, low-input, low-bias construction of shotgun fragment libraries by high-density in vitro transposition. Genome Biol. 11, R119 (2010).
24. Chu C C, and Clark A J. A 10- rather than 9-bp duplication associated with insertion of Tn5 in Escherichia coli K-12. Plasmid 22, 260-264 (1989).
25. Amini S, et al. Haplotype-resolved whole-genome sequencing by contiguity-preserving transposition and combinatorial indexing. Nat Genet. 46, 1343-1349 (2014).
26. Martin M. Cutadapt removes adapter sequences from high-throughput sequencing reads. EMBnet J. 17, 10-12 (2011).

### Informal Sequence Listing:

>sp|Q46731|TN5P_ECOLX Transposase for transposon
Tn5 OS = Escherichia coli OX = 562 GN = tnpA
PE = 1 SV = 1
(SEQ ID NO: 18)
MITSALHRAADWAKSVFSSAALGDPRRTARLVNVAAQLAKYSGKSITISS
EGSEAMQEGAYRFIRNPNVSAEAIRKAGAMQTVKLAQEFPELLAIEDTTS
LSYRHQVAEELGKLGSIQDKSRGWWVHSVLLLEATTFRTVGLLHQEWWMR
PDDPADADEKESGKWLAAAATSRLRMGSMMSNVIAVCDREADIHAYLQDK
LAHNERFVVRSKHPRKDVESGLYLYDHLKNQPELGGYQISIPQKGVVDKR
GKRKNRPARKASLSLRSGRITLKQGNITLNAVLAEEINPPKGETPLKWLL
LTSEPVESLAQALRVIDIYTHRWRIEEFHKAWKTGAGAERQRMEEPDNLE
RMVSILSFVAVRLLQLRESFTLPQALRAQGLLKEAEHVESQSAETVLTPD
ECQLLGYLDKGKRKRKEKAGSLQWAYMAIARLGGFMDSKRTGIASWGALW
EGWEALQSKLDGFLAAKDLMAQGIKI
>sp|Q46731-2|TN5P_ECOLX Isoform 2 of Transposase
for transposon Tn5 OS = Escherichia coli OX = 562
GN = tnpA
(SEQ ID NO: 21)
MQEGAYRFIRNPNVSAEAIRKAGAMQTVKLAQEFPELLAIEDTTSLSYRH
QVAEELGKLGSIQDKSRGWWVHSVLLLEATTFRTVGLLHQEWWMRPDDPA
DADEKESGKWLAAAATSRLRMGSMMSNVIAVCDREADIHAYLQDKLAHNE
RFVVRSKHPRKDVESGLYLYDHLKNQPELGGYQISIPQKGVVDKRGKRKN
RPARKASLSLRSGRITLKQGNITLNAVLAEEINPPKGETPLKWLLLTSEP
VESLAQALRVIDIYTHRWRIEEFHKAWKTGAGAERQRMEEPDNLERMVSI
LSFVAVRLLQLRESFTLPQALRAQGLLKEAEHVESQSAETVLTPDECQLL
GYLDKGKRKRKEKAGSLQWAYMAIARLGGFMDSKRTGIASWGALWEGWEA
LQSKLDGFLAAKDLMAQGIKI

What is claimed is:

1. A method for improving the reconstruction of a single cell genome, comprising:
(i) obtaining genomic DNA derived from a single fully disrupted cell;
(ii) contacting and fragmenting the genomic DNA using a transposase loaded with two identical transposon ends to form a plurality of genomic DNA fragments each labeled with an identical transposon end at its 5′ and 3′ ends;
(iii) extending a complementary strand of each fragment using a universal primer comprising a sequence complementary to the identical transposon end to generate one or more extension products;
(iv) determining the nucleotide sequence between transposon ends of the extension products;
(v) detecting one or more shorter extension products and one longer extension product comprising one identical segment of genomic sequence;
(vi) disregarding the one or more shorter extension products; and
(vii) identifying appropriate connections that facilitate sequence chaining among the remaining extension products based on overlapping unique 8-10 nucleotide sequences immediately next to the transposon end to determine the phase of each sequence, thereby reconstructing the genome.

2. The method of claim 1, wherein step (vii) determines the ploidy of a genomic region in the genome.

3. The method of claim 1, wherein the extension products are linked together in 5′ to 3′ direction by concatenating contiguous fragments at transposon junctions.

4. The method of claim 3, wherein the sequenced extension products are linked according to their unique fragment identifiers (UFI) comprising the start and end nucleotide positions of the fragments.

5. The method of claim 1, wherein the disregarded extension products from step (vi) comprise a sequence complementary to the 5′ end of one or more retained extension products.

6. The method of claim 1, wherein the disregarded extension products from step (vi) comprise a sequence complementary to the 3′ end of one or more retained extension products.

7. The method of claim 1, wherein the disregarded extension products from step (vi) comprise a sequence complementary to both the 5′ and 3′ ends of the one or more retained extension products.

8. The method of claim 5, wherein the one or more retained extension products comprise a neighboring or adjacent in-phase extension product.

9. The method of claim 1, further comprising amplifying the extension products.

10. The method of claim 7, further comprising adding a barcode sequence to the amplified extension products.

11. The method of claim 1, wherein the extension products are bioinformatically linked by concatenating the fragments at transposon junctions.

12. The method of claim 1, wherein the transposon end comprises a universal primer.

13. The method of claim 1, where the single cell genome comprises one or more alleles of at least one genetic locus.

14. The method of claim 1, where the single cell genome comprises two or more chromosomes.

15. The method of claim 1, wherein the single fully disrupted cell is a monoploid cell, diploid cell, a tetraploid cell, a multiploid cell, or a cancer cell.

16. A method for counting two DNA molecules, comprising
(i) obtaining genomic DNA derived from a single fully disrupted cell;
(ii) contacting and fragmenting the genomic DNA using a transposase loaded with two identical transposon ends to form a plurality of genomic DNA fragments each labeled with an identical transposon end at its 5′ and 3′ ends;
(iii) extending a complementary strand of each fragment using a universal primer comprising a sequence complementary to the identical transposon end to generate one or more extension products;
(iv) determining the nucleotide sequence the extension products;
(v) detecting one or more shorter extension products and one longer extension product comprising one identical segment of genomic sequence;
(vi) disregarding the one or more shorter extension products; and
(vii) identifying appropriate connections that facilitate sequence chaining among the remaining extension products based on overlapping unique 8-10 nucleotide sequences immediately next to the transposon end to determine the phase of each sequence to create a contiguous sequence;
(viii) assigning the contiguous sequence to a first or second DNA molecule, thereby counting the DNA molecules.

17. The method of claim 16, wherein the two or more DNA molecules comprise the same or identical nucleic acid sequences.

18. The method of claim 16, wherein the assigning step occurs using the rules of exclusivity and greediness.

19. The method of claim 16, wherein counting the DNA molecules comprises counting digital DNA molecules.

20. A system or device for performing the method of claim 1.

21. The system or device of claim 20, wherein the system or device is a computer system or computerized device.


SEQUENCE LISTING (21 sequences)

SEQ ID NO: 1 - DNA, 136 bp, Artificial Sequence
  Description of sequence The 9-base junction and its surrounding regions of Chr XII/NC_001144.5 755,9 60-775,121
ttggtcacggttggcgagtaaagtacgtctgcagctggcggaatatttgcatacggcttcgccgtttctgccactggtttagtgataactttcttcggaggtgatgcctgataatcctgccgaaatttaccaatta

SEQ ID NO: 2 - DNA, 13 bp, Artificial Sequence
  Description of sequence 9-base canonical cut most preferred base preference
cagttttaaactg

SEQ ID NO: 3 - DNA, 13 bp, Artificial Sequence
  Description of sequence 9-base canonical cut secondary base preference
atcaacagtttaa

SEQ ID NO: 4 - DNA, 13 bp, Artificial Sequence
  Description of sequence 9-base canonical cut secondary base preference
tgaccactgggct

SEQ ID NO: 5 - DNA, 13 bp, Artificial Sequence
  Description of sequence 9-base canonical cut secondary base preference
gctggggcccagc

SEQ ID NO: 6 - DNA, 12 bp, Artificial Sequence
  Description of sequence 8-base non-canonical cut most preferred base preference
cagtttaaacag

SEQ ID NO: 7 - DNA, 12 bp, Artificial Sequence
  Description of sequence 8-base non-canonical cut secondary base preference
gtcccattgttt

SEQ ID NO: 8 - DNA, 12 bp, Artificial Sequence
  Description of sequence 8-base non-canonical cut secondary base preference
ttagggcccgca

SEQ ID NO: 9 - DNA, 12 bp, Artificial Sequence
  Description of sequence 8-base non-canonical cut secondary base preference
actaacggtagc

SEQ ID NO: 10 - DNA, 14 bp, Artificial Sequence
  Description of sequence 10-base non-canonical cut most preferred base preference
cagtattaatactg

SEQ ID NO: 11 - DNA, 14 bp, Artificial Sequence
  Description of sequence 10-base non-canonical cut secondary base preference
atcctaatggggaa

SEQ ID NO: 12 - DNA, 14 bp, Artificial Sequence
  Description of sequence 10-base non-canonical cut secondary base preference
gtcggcgctattct

SEQ ID NO: 13 - DNA, 14 bp, Artificial Sequence
  Description of sequence 10-base non-canonical cut secondary base preference
tctacgcgcccagc

SEQ ID NO: 14 - DNA, 22 bp, Artificial Sequence
  Description of sequence NEX8a  Constructed tranpososome
cagagatgtgtataagagacag

SEQ ID NO: 15 - DNA, 19 bp, Artificial Sequence
  Description of sequence Tn5Up Constructed tranpososome
ctgtctcttatacacatct

SEQ ID NO: 16 - DNA, 33 bp, Artificial Sequence
  Description of sequence Illumina Read1 primer
tcgtcggcagcgtcagatgtgtataagagacag

SEQ ID NO: 17 - DNA, 34 bp, Artificial Sequence
  Description of sequence Illumina Read2 primer
gtctcgtgggctcggagatgtgtataagagacag

SEQ ID NO: 18 - protein, 476 aa, Escherichia coli
MITSALHRAADWAKSVFSSAALGDPRRTARLVNVAAQLAKYSGKSITISSEGSEAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTLPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 19 - DNA, 19 bp, Artificial Sequence
  Description of sequence transferred transposon end
agatgtgtataagagacag

SEQ ID NO: 20 - DNA, 13 bp, Artificial Sequence
  Description of sequence non-transferred transposon end
cttatacacatct

SEQ ID NO: 21 - protein, 421 aa, Escherichia coli
MQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTLPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI